(19) United States
(12) Patent Application Publication
(10) Pub. No.: US 2004/0197784 A1
(43) Pub. Date: Oct. 7, 2004

(54) Retinoid inducible proteins of vascular smooth muscle cells and uses thereof

(76) Inventors: Joseph Michael Miano, Rochester, NY (US); Jeffrey Williams Streb, Rochester, NY (US); Jiyuan Chen, Los Angeles, CA (US)

Correspondence Address: Edwin V Merkel, Nixon Peabody, Clinton Square, PO Box 31051, Rochester, NY, 14603, US

(21) Appl. No.: 10/468,655

(22) PCT Filed: Feb. 22, 2002
(86) PCT No.: PCT/US02/05560

Related U.S. Application Data
(60) Provisional application No. 60/293,097, filed on May 23, 2001.
(60) Provisional application No. 60/271,183, filed on Feb. 22, 2001.

(51) Int. Cl. C12Q 1/68; C12N 9/64; C07H 21/04

(52) U.S. Cl. 435/6; 435/69.1; 435/226; 435/320.1; 435/325; 536/23.2

(57) ABSTRACT

The present invention relates to an isolated retinoid inducible serine carboxypeptidase proteins or polypeptides, and the nucleic acid molecules encoding such a protein or polypeptide. Nucleic acid constructs, expression systems and host cells containing those nucleic acid molecules, and antibodies raised against the proteins or polypeptides are also disclosed. The present invention also relates to methods for detecting a vascular disease or disorder, inhibiting smooth muscle cell growth, treating vascular hyperplasia, and inhibiting the activity of extracellular regulated kinase. The present invention also relates to a transgenic non-human animal lacking a gene encoding a retinoid inducible protein or polypeptide.

The present invention claims benefit of U.S. Provisional Patent Application Serial No. 60/293,097, filed May 23, 2001, and U.S. Provisional Patent Application Serial No. 60/271,183 filed Feb. 22, 2001, each of which is hereby incorporated by reference in its entirety.

This invention was made as a result of research funded by Cardiovascular Research Training Grant No. 1T32HL07949 from the National Institutes of Health. The United States Government may have certain rights in this invention.

### FIELD OF THE INVENTION

The present invention relates to retinoid inducible serine carboxypeptidase proteins or polypeptides isolated from smooth muscle cells, and the nucleic acids that encode such proteins or polypeptides. The present invention also relates to methods of detection and treatment of vascular injury and disease using retinoid inducible proteins or polypeptides including the serine carboxypeptidase proteins or polypeptides disclosed herein.

### BACKGROUND OF THE INVENTION

Vascular smooth muscle cell (“SMC”) activation is a salient feature of several pathological conditions including atherosclerosis, hypertension, vein graft failure, restenosis, and transplant arteriopathy (Libby et al., “A Cascade Model for Restenosis. A Special Case of Atherosclerosis Progression,” Circulation 86: III-47-III-52 (1992); Schwartz et al., “The Intima: Soil For Atherosclerosis and Restenosis,” Circ. Res. 77:445-465 (1995)). These conditions are characterized by the proliferation and subverted differentiation of SMCs with consequent neointimal formation and possible plaque instability. Although the literature is replete with pharmaceutical interventions for experimental neointimal formation, very few therapies have proved widely effective in ameliorating human vascular stenoses. Consequently, complications stemming from neointimal formation continue to be a vexing issue for clinicians.

Much effort has been directed towards the identification and testing of interventions that inhibit SMC growth (Schwartz et al., “The Intima: Soil For Atherosclerosis and Restenosis,” Circ. Res. 77:445-465 (1995)) as well as elucidating the transcriptional program of SMC differentiation (Owens, G. K., “Regulation of Differentiation of Vascular Smooth Muscle Cells,” Physiol. Rev. 75:487-517 (1995)). Considerable progress has been made in the latter case with the cloning and characterization of several SMC-restricted promoters whose utility will likely be appraised in the context of SMC-specific gene targeting for vascular disease. It must be stressed, however, that while many pharmaceutical approaches have proven effective in animal models of vessel disease, their translation into clinical efficacy has been disappointing. Thus, new interventional strategies should be directed towards numerous aspects of vessel wall disease rather than targeting any one pathway or protein.

Retinoids are natural and synthetic derivatives of vitamin A (Sporn et al., “Prevention of Chemical Carcinogenesis by Vitamin A and Its Synthetic Analogs (Retinoids),” Fed. Proc. 35:1332-1338 (1976)) that have myriad effects on cellular growth and differentiation processes (Nau et al., “Retinoids: The Biochemical and Molecular Basis of Vitamin A and Retinoid Action,” Handbook of Experimental Pharmacology Vol. 139, Berlin, Springer-Verlag (2000)). Retinoids have been used clinically for the successful management of several diseases, most notably certain cancers (Hong et al., “Recent Advances in Chemoprevention of Cancer,” Science 278:1073-1077 (1997)). Given the similar events underlying the pathogenesis of neoplasia and such vascular disorders as atherosclerosis and post-injury restenosis (i.e., exuberant cell growth and subverted cellular differentiation), numerous studies have been undertaken to demonstrate the utility of retinoids in conferring a desirable SMC or vascular wall phenotype. For example, retinoids can antagonize growth factor-stimulated SMC proliferation in vitro (Hayashi, et al., “Modulations of Elastin Expression and Cell Proliferation by Retinoids in Cultured Vascular Smooth Muscle Cells,” J. Biochem. 117:132-136 (1995); Miano et al., “Retinoid Receptor Expression and All-Trans Retinoic Acid-Mediated Growth Inhibition in Vascular Smooth Muscle Cells,” Circulation 93:1886-1895 (1996); Chen et al., “Retinoic Acid Uses Divergent Mechanisms to Activate or Suppress Mitogenesis in Rat Aortic Smooth Muscle Cells,” J. Clin. Invest. 102:653-662 (1998); Pakala et al., “RAR y Agonists Inhibit Proliferation of Vascular Smooth Muscle Cells,” J. Cardiovasc. Pharmacol. 35:302-308 (2000); Benson et al., “Ligands For the Peroxisome Proliferator-Activated Receptor-G and the Retinoid X Receptor-a Exert Synergistic Antiproliferative Effects on Human Coronary Artery Smooth Muscle Cells,” Mol. Cell. Biol. Res. Comm. 3:159-164 (2000); Axel et al., “All-Trans Retinoic Acid Regulates Proliferation, Migration, Differentiation, and Extracellular Matrix Turnover of Human Arterial Smooth Muscle Cells,” Cardiovasc. Res. 49:851-862 (2001)), inhibit SMC migration (Axel et al., “All-Trans Retinoic Acid Regulates Proliferation, Migration, Differentiation, and Extracellular Matrix Turnover of Human Arterial Smooth Muscle Cells,” Cardiovasc. Res. 49:851-862 (2001); James et al., “Induction of Collagenase and Stromelysin Gene Expression by Mechanical Injury in a Vascular Smooth Muscle-Derived Cell Line,” J. Cell. Physiol. 157:426-437 (1993)), and promote a more differentiated SMC phenotype (Axel et al., “All-Trans Retinoic Acid Regulates Proliferation, Migration, Differentiation, and Extracellular Matrix Turnover of Human Arterial Smooth Muscle Cells,” Cardiovasc. Res. 49:851-862 (2001); Haller et al., “Differentiation of Vascular Smooth Muscle Cells and the Regulation of Protein Kinase C-alpha,” Circ. Res. 76:21-29 (1995); Gollasch et al., “L-Type Calcium Channel Expression Depends on the Differentiated State of Vascular Smooth Muscle Cells,” FASEB J. 12:593-601 (1998); Wiegman et al., “All-Trans-Retinoic Acid Limits Restenosis After Balloon Angioplasty in the Focally Atherosclerotic Rabbit: A Favorable Effect on Vessel Remodeling,” Arterioscler. Thromb. Vasc. Biol. 20:89-95 (2000)). In a series of complementary in vivo studies, retinoids were shown to minimize vascular narrowing following injury to the vessel wall (Wiegman et al., “All-Trans-Retinoic Acid Limits Restenosis After Balloon Angioplasty in the Focally Atherosclerotic Rabbit: A Favorable Effect on Vessel Remodeling,” Arterioscler. Thromb. Vasc. Biol. 20:89-95 (2000); Miano et al., “All-Trans-Retinoic Acid Reduces Neointimal Formation and Promotes Favorable Geometric Remodeling of the Rat Carotid Artery After Balloon Withdrawal Injury, Circulation 98:1219-1227 (1998); Neuville et al., “Retinoic Acid Regulates Arterial Smooth Muscle Cell Proliferation and Phenotypic Features In Vivo and In Vitro Through an RARa-Dependent Signaling Pathway,” Arterioscler. Thromb. Vasc. Biol. 19:1430-1436 (1999); DeRose et al., “Retinoic Acid Suppresses Intimal Hyperplasia and Prevents Vessel Remodeling Following Arterial Injury,” Cardiovasc. Surg. 7:633-639 (1999); Chen et al., “All-Trans Retinoic Acid Reduces Intimal Thickening After Balloon Angioplasty in Atherosclerotic Rabbits,” Chin. Med. J. 112:121-123 (1999); Lee et al., “All-Trans-Retinoic Acid Attenuates Neointima Formation With Acceleration of Reendothelialization in Balloon-Injured Rat Aorta,” J. Korean Med. Sci. 15:31-36 (2000); Leville et al., “All-Trans-Retinoic Acid Decreases Vein Graft Intimal Hyperplasia and Matrix Metalloproteinase Activity In Vivo,” J. Surg. Res. 90:183-190 (2000)). In a very recent report, rexinoids were demonstrated to reduce atherosclerosis in the apolipoprotein knockout mouse (Claudel et al., “Reduction of Atherosclerosis in Apolipoprotein E Knockout Mice by Activation of the Retinoid X Receptor,” Proc. Natl. Acad. Sci. USA 98:2610-2615 (2001)). Thus, there is mounting evidence to support a possible role for retinoids and rexinoids in the treatment of vascular disease.

Many retinoids exert their pleiotropic effects through the binding and activation of nuclear retinoid receptors. There are two families of retinoid receptors, each of which comprises three distinct genes. The retinoic acid receptors (RAR α, β, and γ) bind all-trans-retinoic acid (“atRA”) and its 9-cis stereoisomer (9cRA), whereas the more weakly expressed retinoid X receptors (RXR α, β, and γ) bind 9cRA (Hong et al., “Recent Advances in Chemoprevention of Cancer,” Science 278:1073-1077 (1997)). Numerous retinoids have been synthesized and tested for receptor selectivity as a means of reducing the side effects associated with natural retinoid therapy. Many of these synthetic retinoids have recently found clinical utility for a number of diseases (Nau et al., “Retinoids: The Biochemical and Molecular Basis of Vitamin A and Retinoid Action,” Handbook of Experimental Pharmacology Vol. 139, Berlin, Springer-Verlag (2000)). Ligand-activated retinoid receptor dimers (preferentially as an RAR-RXR) recognize and bind cis elements (called retinoic acid-response elements) in the genome to activate gene transcription. Identifying retinoid-responsive target genes is critical in defining the molecular actions of these potent, biological response modifiers, although other post-transcriptional processes are likely to play a role as well. In recent years, retinoids have been examined for their influence on vascular SMC growth and differentiation, inasmuch as these processes are thought to be of some relevance in the pathogenesis of vascular occlusive disease (Gardner et al., “Retinoids and Cell Growth in the Cardiovascular System,” Life Sci. 65:1607-1613 (1999); Neuville et al., “Retinoids and Arterial Smooth Muscle Cells,” Arterioscler. Thromb. Vasc. Biol. 20:1882-1888 (2000); Miano and Berk, “Retinoids: Versatile Biological Response Modifiers of Vascular Smooth Muscle Phenotype,” Circ Res. 87:355-362 (2000)). Thus, there is a growing body of in vitro data demonstrating that retinoids antagonize growth factor-stimulated SMC hyperplasia while in some cases promoting a more differentiated SMC phenotype (Gardner et al., “Retinoids and Cell Growth in the Cardiovascular System,” Life Sci. 65:1607-1613 (1999); Neuville et al., “Retinoids and Arterial Smooth Muscle Cells,” Arterioscler. Thromb. Vasc. Biol. 20:1882-1888 (2000); Miano and Berk, “Retinoids: Versatile Biological Response Modifiers of Vascular Smooth Muscle Phenotype,” Circ Res. 87:355-362 (2000)). Because cultured SMCs and the aorta express most of the retinoid receptors and display retinoid receptor activity in vitro (Miano, et al., “Retinoid Receptor Expression and All-Trans Retinoic Acid-Mediated Growth Inhibition in Vascular Smooth Muscle Cells,” Circulation 93:1886-1895 (1996)), it is hypothesized that these observed effects on SMC phenotype are related to retinoid receptor-mediated changes in the SMC transcriptome (Chen et al., “A Novel Retinoid-Response Gene Set in Vascular Smooth Muscle Cells,” Biochem. Bio hys. Res. Commun. 281:475482 (2001)). Indeed, studies using retinoid receptor-selective agonists with reduced toxicity have shown inhibitory effects on SMC growth (Chen et al., “Retinoic Acid Uses Divergent Mechanisms to Activate or Suppress Mitogenesis in Rat Aortic Smooth Muscle Cells,” J. Clin. Invest. 102:653-662 (1998); Neuville et al., “A. Retinoic Acid Regulates Arterial Smooth Muscle Cell Proliferation and Phenotypic Features In Vivo and In Vitro Through an Rara-Dependent Signaling Pathway,” Arterioscler. Thromb. Vasc. Biol. 19:1430-1436 (1999); Pakala et al., “RAR y Agonists Inhibit Proliferation of Vascular Smooth Muscle Cells,” J. Cardiovasc. Pharmacol. 35:302-308 (2000)).

The identification of retinoid-response genes in SMC is now needed to provide a greater understanding of the mechanisms underlying retinoid-induced changes in SMC and vessel wall phenotype, and to provide preventive and therapeutic treatment of the many disease conditions and disorders related to SMC proliferation.

The present invention is directed to overcoming these and other deficiencies in the art.

### SUMMARY OF THE INVENTION

The present invention relates to an isolated vertebrate retinoid inducible serine carboxypeptidase protein or polypeptide, more preferably an isolated mammalian retinoid inducible serine carboxypeptidase protein or polypeptide.

The present invention also relates to an isolated nucleic acid molecule encoding a vertebrate retinoid inducible serine carboxypeptidase protein or polypeptide. Expression vectors and host cells containing such a nucleic acid molecule are also disclosed.

The present invention also relates to a nucleic acid construct having a nucleic acid encoding a vertebrate retinoid inducible serine carboxypeptidase. Expression vectors and host cells containing such a nucleic acid construct are also disclosed.

The present invention also relates to a method of detecting presence, absence, or changes in progression or regression of a vascular disease or disorder in a subject which includes: contacting a tissue or fluid sample from a subject with a nucleic acid molecule according to the present invention, or a fragment thereof, as a primer or a probe in a gene amplification detection procedure; and detecting any reaction which indicates amplification of a target with the primer or probe, where amplification of the target indicates the presence of a vascular disease or disorder and the lack thereof indicates the absence of the vascular disease or disorder.

The present invention also relates to another method of detecting presence, absence, or changes in progression or regression of a vascular disease or disorder in a subject. This method includes contacting a tissue or fluid sample from a subject with a nucleic acid molecule according to the present invention, or a fragment thereof, as a probe, under conditions effective to cause hybridization between the probe and a target to form a hybridization complex; and determining whether any hybridization complex forms during said contacting, where formation of a hybridization complex indicates the presence of a vascular disease or disorder and lack thereof indicates the absence of the vascular disease or disorder.

The present invention also relates to an isolated antibody or binding portion thereof which binds to a vertebrate retinoid inducible serine carboxypeptidase protein or polypeptide of the present invention.

The present invention also relates to another method of detecting presence, absence, or changes in progression or regression of a vascular disease or disorder in a subject. This method involves contacting a tissue or fluid sample from a subject with an antibody or binding portion of the present invention under conditions effective to permit formation of an antigen-antibody/binding portion complex; and determining whether the antigen-antibody/binding portion complex has formed using an assay system, where the presence of antigen-antibody/binding portion complex indicates the presence of a vascular disease or disorder and the lack thereof indicates the absence of the vascular disease or disorder.

The present invention also relates to a method of inhibiting smooth muscle cell growth which includes increasing the intracellular concentration of a retinoid-inducible protein or polypeptide in a smooth muscle cell under conditions effective to inhibit the growth of the smooth muscle cell.

The present invention also relates to a method of treating vascular hyperplasia. This involves increasing the intracellular concentration of a retinoid-inducible protein or polypeptide in vascular smooth muscle cells at a site of vascular hyperplasia under conditions effective to treat the vascular hyperplasia.

The present invention also relates to a method of inhibiting the activity of extracellular regulated kinase which includes contacting an extracellular regulated kinase with a retinoid-inducible protein or polypeptide under conditions effective to inhibit the activity of the extracellular regulated kinase.

The present invention also relates to a transgenic non-human animal whose somatic and germ cells lack a gene encoding a retinoid-inducible protein or polypeptide, or possess a disruption in that gene, whereby the animal exhibits increased smooth muscle cell growth and neointimal formation following vascular trauma as compared to non-transgenic animals.

The present invention demonstrates the ability of RISC to inhibit vascular smooth muscle cell proliferation, which offers numerous therapeutic and preventative treatments to vascular hyperplasia. Diagnostic monitoring for the presence, absence, or changes in the progression or regression of vascular diseases or disorders are also provided. Moreover, through the construction of a transgenic “knockout” animal will reveal appropriate expression of the endogenous RISC gene through an integrated lacZ reporter gene commonly used in knockout mice. Inactivation of the RISC locus should assist in assigning other function to the protein. Based on the results reported herein, it is believed that such knockout animals will provide an in vivo model system to study RISC-associated disease.

### BRIEF DESCRIPTION OF THE DRAWINGS

FIG. 1 shows a sequence comparison of RISC's putative serine carboxypeptidase (“SC”) substrate binding domain (1) and catalytic domains (II-IV) for rat SEQ ID No: 36, human SEQ ID No: 40, and mouse SEQ ID No: 38 RISC as compared to five known SCs. The five known SC's are A. aegypti VCP (P42660), Human Protective Protein (NP_{—}000299), C. elegans F41C3.5 (U23521), H. vulgare Carboxypeptidase C (Y09604), and S. cerevisiae Carboxypeptidase Y (NP_{—}014026). C. elegans F22E12.1 (T21275) and D. melanogaster CG3344 (AAF47405) are two putative SCs with homology to RISC. Boxed residues represent invariant amino acids in the conserved domains. Asterisk (*) indicates critical residues in the serine carboxypeptidase Ser-Asp-His catalytic triad. For SC domain I: human, rat, and mouse RISC (SEQ ID No: 1), C. elegans F22E12.1 (SEQ ID No: 2), D. melanogaster CG3344 and A. aegypti VCP (SEQ ID No: 3), human protective protein (cathepsin A) and S. cerevisiae Cpd Y (SEQ ID No: 4), C. elegans F41C3.5 (SEQ ID No: 5), H. vulgare Cpd C (SEQ ID No: 6), and consensus for these sequences (SEQ ID No: 7). For catalytic domain II: human, rat, and mouse RISC (SEQ ID No: 8), C. elegans F22E12.1 and D. melanogaster CG3344 (SEQ ID No: 9), A. aegypti VCP (SEQ ID No: 10), human protective protein (cathepsin A) (SEQ ID No: 11), C. elegans F41C3.5 (SEQ ID No: 12), H. vulgare Cpd C (SEQ ID No: 13), S. cerevisiae Cpd Y (SEQ ID No: 14), and consensus for these sequences (SEQ ID No: 15). For catalytic domain III: human, rat, and mouse RISC (SEQ ID No: 16), C. elegans F22E12.1 (SEQ ID No: 17), D. melanogaster CG3344 (SEQ ID No: 18), A. aegypti VCP (SEQ ID No: 19), human protective protein (cathepsin A) (SEQ ID No: 20), C. elegans F41C3.5 (SEQ ID No: 21), H. vulgare Cpd C (SEQ ID No: 22), S. cerevisiae Cpd Y (SEQ ID No: 23), and the consensus for these sequences (SEQ ID No: 24). For catalytic domain IV: rat RISC (SEQ ID No: 25), human and mouse RISC (SEQ ID No: 26), C. elegans F22E12.1 (SEQ ID No: 27), D. melanogaster CG3344 (SEQ ID No: 28), A. aegypti VCP (SEQ ID No: 29), human protective protein (cathepsin A) (SEQ ID No: 30), C. elegans F41C3.5 (SEQ ID No: 31), H. vulgare Cpd C (SEQ ID No: 32), S. cerevisiae Cpd Y (SEQ ID No: 33), and the consensus for these sequences (SEQ ID No: 34)

FIG. 2 shows the Rattus norvegicus RISC nucleotide (SEQ ID No: 37) and amino acid (SEQ ID No: 36) sequences. Capital letters represent coding sequence; lower case letters represent 5′ and 3′ untranslated regions (UTR). The boxed amino acids at the N-terminus represent the signal peptide; the cleavage site is predicted between amino acids 28 and 29. The four heavy underlined regions of the amino acid sequences are conserved motifs common to serine carboxypeptidases; the italicized letters in each are residues of the Ser-Asp-His catalytic triad. The lightly underlined region in the 3′ UTR is a consensus polyadenylation sequence.

FIGS. 3A-B show typical slot blots arrayed with random subtracted “tester” cDNA clones. FIG. 3A shows a blot hybridized to radiolabeled tester (atRA-treated) cDNA. FIG. 3B shows a blot hybridized to radiolabeled driver (control) cDNA. Arrows depict tRA-induced Stoned B/TFIIAα/β-like factor (“SALF”) (col. B4) and LDH-B (D10).

FIG. 4 is a representative time-course study showing expression of immediate early retinoid response genes. Rat aortic smooth muscle cells (“RASMC”) were stimulated with atRA for the indicated times and then processed for total RNA isolation and Northern blotting. Two duplicate blots were then sequentially hybridized to each of the 14 cDNAs. Scanning densitometry was performed at each time point and normalized to the corresponding GAPDH signal to ascertain the peak mRNA induction value listed in Table 2.

FIG. 5 is a representative time-course study showing expression of delayed retinoid-response genes, carried out as described above in FIG. 4.

FIG. 6 shows a representative Northern blot showing cycloheximide (“CHX”) effects on atRA inducible gene expression. RASMC were pre-incubated for 10 minutes in the absence (−) or presence (+) of 2.5 μg/ml CHX, stimulated for the indicated times with 2×10⁻⁶ mol/L atRA, and then processed for total RNA isolation and Northern blotting. All 4 genes show atRA-induced mRNA expression after 24 hr stimulation. The mRNA induction of SSAT and α₈ integrin is completely blocked at 24 hr with CHX. SALF and Src-Suppressed C Kinase Substrate (“SSeCKS”) mRNA induction, however, is protein synthesis-independent. Refer to Table 2 for a complete summary of the CHX sensitivity studies.

FIGS. 7A-B shows a representative Northern blot of two retinoid-responsive genes in various rat tissues. Indicated rat tissues were isolated for total RNA and gel fractionated for Northern blotting. The blot was sequentially hybridized to rat tTG, FIG. 7A, and rat α₈ integrin, FIG. 7B. An 18 S probe was used to demonstrate equivalent RNA loading. The positions of the major ribosomal RNA makers are shown to the right.

FIGS. 8A-F show the spatial expression of tTG and α₈ integrin mRNA in the vessel wall. Adjacent sections (A-B and C-F) obtained from rat carotid arteries were prepared for in situ hybridization as described in the methods. Sense riboprobes to α₈ integrin (panel A), SM22 panel C) and tTG reveal low background hybridization across the vessel wall. In contrast, antisense riboprobes to α₈ integrin (panel B) and tTG (panel F) show a prominent hybridization signal that co-distributes with an SM22 riboprobe (panel D). Panel E shows a brightfield image of the section shown in panel F. The scale bar underneath panel F is 200 μm.

FIGS. 9A-B show the induction of retinoid inducible serine carboxypeptidase (“RISC”) mRNA expression by atRA in a time-dependent manner. Total RNA isolated from RASMC treated with 2.0 μM atRA for 0, 3, 6, 12, 24, 48, 72, 96, 120 h was analyzed by Northern blotting with a rat RISC cDNA probe. FIG. 9A shows results with fresh atRA used every 24 h. FIG. 9B shows results over 5 days with one application of the same dose of atRA. Blots were also probed with GAPDH as a control for equal loading for each lane. Data shown are representative of two independent experiments.

FIG. 10 shows the in vitro transcription and translation of RISC cDNA. A RISC cDNA fragment corresponding to the coding sequence was cloned into pBluescript and in vitro transcription and translation of the resulting circular plasmid was performed with ³⁵S-methionine as described in the examples infra. Molecular size markers are indicated at the left side. A luciferase cDNA was included as a control and point of reference.

FIGS. 11A-B show the expression of a rat RISC-His tagged fusion protein in COS-7 cells detected by immunofluorescence microscopy. FIG. 11A shows mock-transfected COS-7 cells. FIG. 11B shows the COS-7 cells transfected with the fusion protein. Note the perinuclear accumulation of RISC-His in the right panel.

FIG. 12 shows Western blotting results of COS-7 cells transfected transiently with RISC-His (lanes 1 and 3) or mock-transfected with empty His plasmid (lanes 2 and 4). CM refers to the conditioned medium and CL denotes cellular lysate. Note the shift in size of the immunoreactive products (as compared to in vitro translated RISC in FIG. 10) due to the His residues and probable post-translational modifications (e.g., N-linked glycosylation).

FIG. 13 shows the tissue-restricted expression of rat RISC mRNA. Total RNA isolated from various normal rat tissues were subjected to Northern hybridization using a rat RISC cDNA probe. The transcript for rat RISC was detected in aorta, bladder, and kidney with low level expression in heart, lung, spleen, and stomach. 18 S ribosomal RNA was also probed and bands used as controls for equal loading of total RNA for each tissue.

FIG. 14 shows the tissue-restricted expression of human RISC. Poly A+ RNA from human tissues were subjected to Northern hybridization using a human RISC cDNA probe. The human RISC transcript was highly enriched in kidney and heart. β-actin mRNA demonstrates relatively equal mRNA loading in the human blot. The lower band obtained with the β3-actin probe in skeletal muscle probably represents cross-hybridization to the skeletal muscle α-actin mRNA. “PBL” represents peripheral blood leukocyte mRNA.

FIGS. 15A-H show an in situ hybridization analysis of RISC mRNA expression in rat tissues. FIGS. 15A-B are sections obtained from aorta; FIGS. 15C-D are obtained from bladder; and FIGS. 15E-H are obtained from kidney. All sections were prepared for in situ hybridization with antisense (A, C, E, and G-H) or sense (B, D, and F) RISC riboprobes. Panels A-F represent darkfield microscopic images. Sense RISC exhibited only background hybridization. FIG. 15A shows a modest increase in RISC mRNA observed in the tunica media of the rat aorta Expression of RISC appears to be enriched in the transitional epithelium of the bladder, seen in FIG. 15C. In FIG. 15E, note the restricted expression of RISC to the renal cortex with little or no signal in the underlying medulla The punctate regions of the cortex devoid of hybridization signal in panel E represent glomeruli. High magnification brightfield microscopy (G-H) shows that RISC is restricted to the cuboidal epithelium of the proximal convoluted tubules. Abbreviations are m, renal medulla; d, distal convoluted tubule; g, glomerulus; and p, proximal convoluted tubule. Magnifications are 20× (for A-F) and 600× (for G-H).

FIG. 16 shows chromosomal mapping of rat RISC. Rat RISC was mapped to rat chromosome 10q using a radiation hybrid panel. Numbers to the left side of the map represent map distances in centi-rays. This region of rat chromosome 10q is syntenic with human chromosome 17q23.1, which is where the human RISC gene has been mapped.

FIG. 17 is a graph illustrating the effects of RISC on stably transfected PAC1 SMC growth.

FIG. 18 is an image of an immunoblot using phospho-specific antibodies for pERK in stably transfected PAC1 SMC induced with serum or a purified growth factor.

### DETAILED DESCRIPTION OF THE INVENTION

The present invention relates generally to the use of any one of several retinoid inducible genes, or their encoded proteins or polypeptides for the prevention and therapeutic regulation of smooth muscle cell growth and vascular hyperplasia as well as other uses which are disclosed herein.

One aspect of the present invention relates to an isolated vertebrate retinoid inducible serine carboxypeptidase (“RISC”) protein or polypeptide, as well as an isolated nucleic acid molecule encoding the vertebrate RISC protein or polypeptide. The nucleic acid can be either DNA or RNA. Preferably, the vertebrate RISC protein or polypeptide is a mammalian RISC protein or polypeptide. By “mammalian,” it is intended to encompass all mammalian RISC proteins or polypeptides, but preferably, human, rat, and mouse.

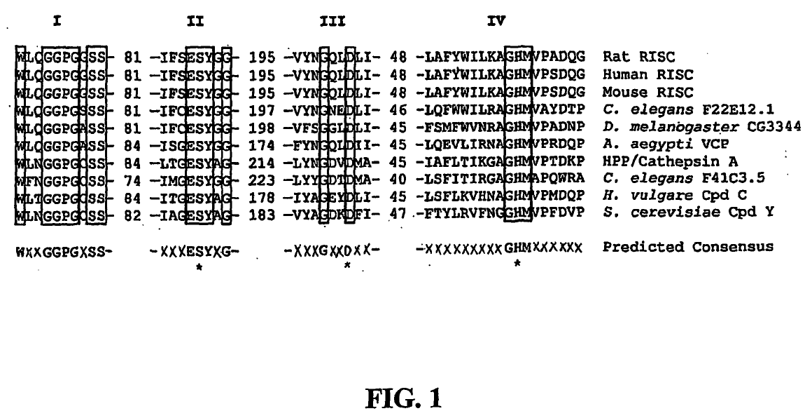

Serine carboxypeptidases (EC 3.4.16.1) are a family of lysosomal glycoproteins (45-75 kDa) that exhibit carboxy-terminal proteolytic activity at acidic pH (Remington et al., “Carboxypeptidases C and D,” Methods Enzymol. 244:231-248 (1994), which is hereby incorporated by reference in its entirety). Serine carboxypeptidases (SC) share a number of structural features including a signal sequence for intracellular trafficking and/or secretion, multiple N-linked glycosylation sites, and four evolutionarily conserved domains involved with substrate binding and catalysis, as shown in FIG. 1. One or more of these domains is present in the amino acid sequence of the RISC protein or polypeptide of the present invention. Thus, the RISC protein or polypeptide of the present invention preferably has one or more domains selected from the group of (a) a serine carboxypeptidase substrate binding domain with an amino acid sequence of WXXGGPGXSS (SEQ ID No: 7) where X is any amino acid; (b) a first catalytic domain with an amino acid sequence of XXXESYXG (SEQ ID No: 15) where X is any amino acid; (c) a second catalytic domain with an amino acid sequence of XXXGXXDLI (SEQ ID No: 24) where X is any amino acid; and (d) a third catalytic domain with an amino acid sequence of XXXXGH (SEQ ID No: 34) where X is any amino acid.

Preferred mammalian RISC proteins or polypeptides of the present invention will include all four of the above-noted domains. More preferably, such mamnalian RISC proteins or polypeptides will include a RISC protein or polypeptide which includes: a serine carboxypeptidase domain with an amino acid sequence of WLQGGPGGSS, (SEQ ID No: 1), a first catalytic domain with an amino acid sequence of IFSESYGG, (SEQ ID No: 8), a second catalytic domain with an amino acid sequence of VYNGQLDLI, (SEQ ID No: 16), and a third catalytic domain with an amino acid sequence of LAFYWILKAGHMVPXDQG, (SEQ ID No: 35) where X is A or S.

An exemplary RISC protein or polypeptide of the present invention is a RISC protein isolated from rat having an amino acid sequence corresponding to SEQ ID No: 36 as follows:

```
Met Glu Leu Ser Arg Arg Ile Cys Leu Val Arg Leu Trp Leu Leu Leu
    1 5 10 15
Leu Ser Phe Leu Leu Gly Phe Ser Ala Gly Ser Ala Leu Asn Trp Arg
                          20 25 30
Glu Gln Glu Gly Lys Glu Val Trp Asp Tyr Val Thr Val Arg Glu Asp
                  35 40 45
Ala Arg Met Phe Trp Trp Leu Tyr Tyr Ala Thr Asn Pro Cys Lys Asn
          50 55 60
Phe Ser Glu Leu Pro Leu Val Met Trp Leu Gln Gly Gly Pro Gly Gly
  65 70 75 80
Ser Ser Thr Gly Phe Gly Asn Phe Glu Glu Ile Gly Pro Leu Asp Thr
                                    85 90 95
Arg Leu Lys Pro Arg Asn Thr Thr Trp Leu Gln Trp Ala Ser Leu Leu
                        100 105 110
Phe Val Asp Asn Pro Val Gly Thr Gly Phe Ser Tyr Val Asn Thr Thr
                115 120 125
Asp Ala Tyr Ala Lys Asp Leu Asp Thr Val Ala Ser Asp Met Met Val
        130 135 140
Leu Leu Lys Ser Phe Phe Asp Cys His Lys Glu Phe Gln Thr Val Pro
145 150 155 160
Phe Tyr Ile Phe Ser Glu Ser Tyr Gly Gly Lys Met Ala Ala Gly Ile
                                165 170 175
Ser Leu Gln Leu His Lys Ala Ile Gln Gln Gly Thr Ile Lys Cys Asn
                        180 185 190
Phe Ser Gly Val Ala Leu Gly Asp Ser Trp Ile Ser Pro Val Asp Ser
                195 200 205
Val Leu Ser Trp Gly Pro Tyr Leu Tyr Ser Val Ser Leu Leu Asp Asn
        210 215 220
Lys Gly Leu Ala Glu Val Ser Asp Ile Ala Glu Gln Val Leu Asn Ala
225 230 235 240
Val Asn Lys Gly Phe Tyr Lys Glu Ala Thr Gln Leu Trp Gly Lys Ala
                                245 250 255
Glu Met Ile Ile Glu Lys Asn Thr Asp Gly Val Asn Phe Tyr Asn Ile
                        260 265 270
Leu Thr Lys Ser Thr Pro Asp Thr Ser Met Glu Ser Ser Leu Glu Phe
                275 280 285
Phe Arg Ser Pro Leu Val Arg Leu Cys Gln Arg His Val Arg His Leu
        290 295 300
Gln Gly Asp Ala Leu Ser Gln Leu Met Asn Gly Pro Ile Lys Lys Lys
305 310 315 320
Leu Lys Ile Ile Pro Asp Asp Val Ser Trp Gly Ala Gln Ser Ser Ser
                                325 330 335
Val Phe Ile Ser Met Glu Glu Asp Phe Met Lys Pro Val Ile Asp Ile
                        340 345 350
Val Asp Thr Leu Leu Glu Leu Gly Val Asn Val Thr Val Tyr Asn Gly
                355 360 365
Gln Leu Asp Leu Ile Val Asp Thr Ile Gly Gln Glu Ser Trp Val Gln
        370 375 380
Lys Leu Lys Trp Pro Gln Leu Ser Arg Phe Asn Gln Leu Lys Trp Lys
385 390 395 400
Ala Leu Tyr Thr Asn Pro Lys Ser Ser Glu Thr Ser Ala Phe Val Lys
                                405 410 415
Ser Tyr Glu Asn Leu Ala Phe Tyr Trp Ile Leu Lys Ala Gly His Met
                        420 425 430
Val Pro Ala Asp Gln Gly Asp Met Ala Leu Lys Met Met Arg Leu Val
                435 440 445
Thr Gln Gln Glu
        450
```

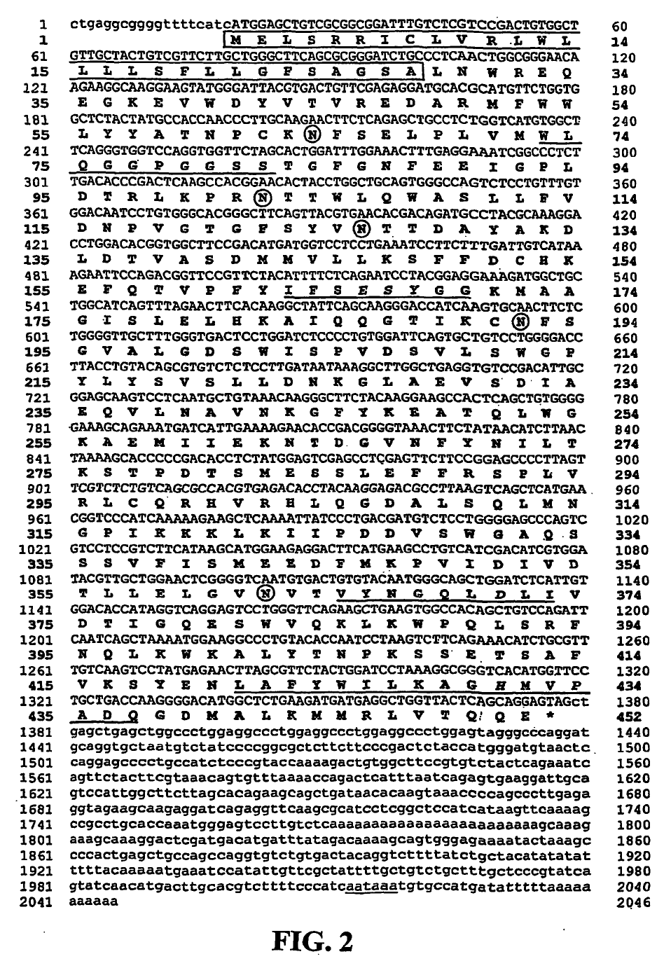

This rat RISC sequence is also shown in FIG. 2, where the four heavy underlined regions of the amino acid sequence are the conserved motifs common to serine carboxypeptidases. The SC binding site is found at amino acids 73-82. The first catalytic domain is found at amino acids 163-170. The second catalytic domain is found at amino acids 365-373. The third catalytic domain is found at amino acids 421-437. In addition, several putative N-linked glycosylation sites are found in the rat RISC primary amino acid sequence. These are shown as circled residues in FIG. 2. The rat RISC has a calculated molecular mass of about 51.2 kDa and a predicted pI of 5.37.

One nucleic acid encoding the rat RISC protein or polypeptide of the present invention is a DNA molecule having a nucleotide sequence corresponding to SEQ ID No: 37 as follows:

ctgaggcggg gttttcatca tggagctgtc gcggcggatt tgtctcgtcc gactgtggct 60 gttgctactg tcgttcttgc tgggcttcag cgcgggatct gccctcaact ggcgggaaca 120 agaaggcaag gaagtatggg attacgtgac tgttcgagag gatgcacgca tgttctggtg 180 gctctactat gccaccaacc cttgcaagaa cttctcagag ctgcctctgg tcatgtggct 240 tcagggtggt ccaggtggtt ctagcactgg atttggaaac tttgaggaaa tcggccctct 300 tgacacccga ctcaagccac ggaacactac ctggctgcag tgggccagtc tcctgtttgt 360 ggacaatcct gtgggcacgg gcttcagtta cgtgaacacg acagatgcct acgcaaagga 420 cctggacacg gtggcttccg acatgatggt cctcctgaaa tccttctttg attgtcataa 480 agaattccag acggttccgt tctacatttt ctcagaatcc tacggaggaa agatggctgc 540 tggcatcagt ttagaacttc acaaggctat tcagcaaggg accatcaagt gcaacttctc 600 tggggttgct ttgggtgact cctggatctc ccctgtggat tcagtgctgt cctggggacc 660 ttacctgtac agcgtgtctc tccttgataa taaaggcttg gctgaggtgt ccgacattgc 720 ggagcaagtc ctcaatgctg taaacaaggg cttctacaag gaagccactc agctgtgggg 780 gaaagcagaa atgatcattg aaaagaacac cgacggggta aacttctata acatettaac 840 taaaagcacc cccgacacct ctatggagtc gagcctcgag ttcttccgga gccccttagt 900 tcgtctctgt cagcgccacg tgagacacct acaaggagac gccttaagtc agctcatgaa 960 cggtcccatc aaaaagaagc tcaaaattat ccctgacgat gtctcctggg gagcccagtc 1020 gtcctccgtc ttcataagca tggaagagga cttcatgaag cctgtcatcg acatcgtgga 1080 tacgttgctg gaactcgggg tcaatgtgac tgtgtacaat gggcagctgg atctcattgt 1140 ggacaccata ggtcaggagt cctgggttca gaagctgaag tggccacagc tgtccagatt 1200 caatcagcta aaatggaagg ccctgtacac caatcctaag tcttcagaaa catctgcgtt 1260 tgtcaagtcc tatgagaact tagcgttcta ctggatccta aaggcgggtc acatggttcc 1320 tgctgaccaa ggggacatgg ctctgaagat gatgaggctg gttactcagc aggagtagct 1380 gagctgagct ggccctggag gccctggagg ccctggaggc cctggagtag ggcccaggat 1440 gcaggtgcta atgtctatcc ccggcgctct tcttcccgac tctaccatgg gatgtaactc 1500 caggagcccc tgccatctcc cgtaccaaaa gactgtggct tccgtgtcta ctcagaaatc 1560 agttctactt cgtaaacagt gtttaaaacc agactcattt aatcagagtg aaggattgca 1620 gtccattggc ttcttagcac agaagcagct gataacacaa gtaaacccca gcccttgaga 1680 ggtagaagca agaggatcag aggttcaagc gcatcctcgg ctccatcata agttcaaaag 1740 ccgcctgcac caaatgggag tccttgtctc aaaaaaaaaa aaaaaaaaaa aaaagcaaag 1800 aaagcaaagg actcgatgac atgatttata gacaaaagca gtgggagaaa atactaaagc 1860 cccactgagc tgccagccag gtgtctgtga ctacaggtct tttatctgct acatatatat 1920 ttttacaaaa atgaaatcca tattgttcgc tattttgctg tctgctttgc tcccgtatca 1980 acatgacttg cacgtctttt cccatcaata aatgtgccat gatattttta aaaaaaaaaa 2040 aaaaaa 2046

The above rat RISC cDNA contains a 452 amino acid open reading frame (nt 19-1375). An in-frame stop codon upstream of a Kozak methionine residue precedes a signal sequence (Stroud et al., “Signal Sequence Recognition and Protein Targeting,” Curr. Opin. Struct. Biol. 9:754-759 (1999), which is hereby incorporated by reference in its entirety) having a possible cleavage site at Ala28, shown as boxed amino acids in FIG. 2. This cDNA sequence has been deposited with GenBank as Accession No. AF330051, which is hereby incorporated by reference in its entirety.

Another exemplary RISC protein or polypeptide of the present invention is a RISC protein isolated from mouse having an amino acid sequence corresponding to SEQ ID No: 38 as follows:

```
Met Glu Leu Ser Arg Arg Ile Cys Leu Val Arg Leu Trp Leu Leu Leu
    1 5 10 15
Leu Ser Phe Leu Leu Gly Phe Ser Ala Gly Ser Ala Ile Asp Trp Arg
                          20 25 30
Glu Pro Glu Gly Lys Glu Val Trp Asp Tyr Val Thr Val Arg Lys Asp
                  35 40 45
Ala His Met Phe Trp Trp Leu Tyr Tyr Ala Thr Asn Pro Cys Lys Asn
          50 55 60
Phe Ser Glu Leu Pro Leu Val Met Trp Leu Gln Gly Gly Pro Gly Gly
  65 70 75 80
Ser Ser Thr Gly Phe Gly Asn Phe Glu Glu Ile Gly Pro Leu Asp Thr
                                  85 90 95
Gln Leu Lys Pro Arg Asn Thr Thr Trp Leu Gln Trp Ala Ser Leu Leu
                        100 105 110
Phe Val Asp Asn Pro Val Gly Thr Gly Phe Ser Tyr Val Asn Thr Thr
                115 120 125
Asp Ala Tyr Ala Lys Asp Leu Asp Thr Val Ala Ser Asp Met Met Val
        130 135 140
Leu Leu Lys Ser Phe Phe Asp Cys His Lys Glu Phe Gln Thr Val Pro
145 150 155 160
Phe Tyr Ile Phe Ser Glu Ser Tyr Gly Gly Lys Met Ala Ala Gly Ile
                                165 170 175
Ser Val Glu Leu Tyr Lys Ala Val Gln Gln Gly Thr Ile Lys Cys Asn
                        180 185 190
Phe Ser Gly Val Ala Leu Gly Asp Ser Trp Ile Ser Pro Val Asp Ser
                195 200 205
Val Leu Ser Trp Gly Pro Tyr Leu Tyr Ser Met Ser Leu Leu Asp Asn
        210 215 220
Gln Gly Leu Ala Met Val Ser Asp Ile Ala Glu Gln Val Leu Asp Ala
225 230 235 240
Val Asn Lys Gly Phe Tyr Lys Glu Ala Thr Gln Leu Trp Gly Lys Ala
                                245 250 255
Glu Met Ile Ile Glu Lys Asn Thr Asp Gly Val Asn Phe Tyr Asn Ile
                        260 265 270
Leu Thr Lys Ser Ser Pro Glu Lys Ala Met Glu Ser Ser Leu Glu Phe
                275 280 285
Leu Arg Ser Pro Leu Val Arg Leu Cys Gln Arg His Val Arg His Leu
        290 295 300
Gln Gly Asp Ala Leu Ser Gln Leu Met Asn Gly Pro Ile Lys Lys Lys
305 310 315 320
Leu Lys Ile Ile Pro Glu Asp Ile Ser Trp Gly Ala Gln Ala Ser Tyr
                                325 330 335
Val Phe Leu Ser Met Glu Gly Asp Phe Met Lys Pro Ala Ile Asp Val
                        340 345 350
Val Asp Lys Leu Leu Ala Ala Gly Val Asn Val Thr Val Tyr Asn Gly
                355 360 365
Gln Leu Asp Leu Ile Val Asp Thr Ile Gly Gln Glu Ser Trp Val Gln
        370 375 380
Lys Leu Lys Trp Pro Gln Leu Ser Lys Phe Asn Gln Leu Lys Trp Lys
385 390 395 400
Ala Leu Tyr Thr Asp Pro Lys Ser Ser Glu Thr Ala Ala Phe Val Lys
                                405 410 415
Ser Tyr Glu Asn Leu Ala Phe Tyr Trp Ile Leu Lys Ala Gly His Met
                        420 425 430
Val Pro Ser Asp Gln Gly Glu Met Ala Leu Lys Met Met Lys Leu Val
                435 440 445
Thr Lys Gln Glu
        450
```

The conserved motifs common to serine carboxypeptidases are also found in the mouse RISC protein sequence. The SC binding site is found at amino acids 73-82. The first catalytic domain is found at amino acids 163-170. The second catalytic domain is found at amino acids 365-373. The third catalytic domain is found at amino acids 421-437.

One nucleic acid encoding the mouse RISC protein or polypeptide of the present invention is a DNA molecule having a nucleotide sequence corresponding to SEQ ID No: 39 as follows:

| | |
|---|---|
| ggttgctgat gttcggcggg gttttcatca tggagctctc gcggcggatc tgtctcgtgc | 60 |
| gactgtggct gctgctccta tcgttcttac tgggcttcag cgcgggatct gccatcgact | 120 |
| ggcgggaacc cgaaggcaag gaagtatggg attatgtgac tgtccgaaag gatgcccaca | 180 |
| tgttctggtg gctctattat gccaccaacc cttgcaagaa cttttcagag ctgcccctgg | 240 |
| tcatgtggct tcagggtggt ccgggtggtt ctagcactgg atttggaaac tttgaggaaa | 300 |
| tcggccctct tgacacccaa ctcaagcctc gaaataccac ctggctgcag tgggccagtc | 360 |
| tcctgtttgt ggataatccc gtgggcacgg gcttcagcta cgtcaacaca acagatgcct | 420 |
| acgcaaagga cctggacacg gtggcttccg acatgatggt tctcctgaaa tccttctttg | 480 |
| attgccataa agaattccag acggttccat tctacatttt ctcagaatcc tacggaggaa | 540 |
| agatggctgc tggcatcagt gtagaacttt acaaggctgt tcagcaaggg accattaagt | 600 |
| gcaacttttc tggggttgct ttgggtgact cctggatctc ccccgtggat tcagtgctgt | 660 |
| cctggggacc ttacctgtat agtatgtctc tccttgataa tcaaggcttg gcgatggtgt | 720 |
| ccgacattgc agagcaagtc ctcgatgctg taaacaaggg cttctacaag gaggccactc | 780 |
| agctgtgggg gaaagcagaa atgatcattg aaaagaacac cgacggggta aacttctata | 840 |
| acatcttaac taaaagcagc ccggagaaag ctatggaatc gagcctcgag ttcctccgga | 900 |
| gccccttagt tcgtctctgt cagcgccatg tgagacacct gcaaggagac gccttaagtc | 960 |
| aactcatgaa cggccccatc aaaaagaagc tcaaaattat ccctgaggat atctcctggg | 1020 |
| gagcccaggc atcttatgtc ttcctaagca tggaagggga cttcatgaag cctgccatcg | 1080 |
| acgttgtgga taagttgctg gcagctgggg tcaatgtgac cgtgtacaac ggacagctgg | 1140 |
| atctcattgt ggacaccata ggtcaggagt cctgggttca gaagctcaag tggccacagc | 1200 |
| tgtccaaatt caatcagcta aaatggaagg ccctgtacac cgatcctaag tcttcagaaa | 1260 |
| cagctgcgtt cgtcaagtcc tatgagaacc tagccttcta ctggatccta aaggccggtc | 1320 |
| acatggttcc ttctgaccaa ggggagatgg ccctgaagat gatgaagctg gtgaccaagc | 1380 |
| aggagtagct gagctggctg gccctggagg cgctaagagc agagcccaga atgcaggtgc | 1440 |
| taatgtctat ccctggtgct cttctcccct gctctgccat gggatatgac tctgggagca | 1500 |
| cctgctctct cccgtaccga aagactgtgg ccttctgtgt ctacttagaa atcagttctg | 1560 |
| cttcccaaag agtatttaaa accagactca tttaatcaaa gtgaagggtt gcaatcgatt | 1620 |
| ggtctcttac tacaaaagca gttgatagca catgtaaatc caagcacttg agaggtagaa | 1680 |
| gaagcaagag gatggatgag aggttcaaac gcatccgcag ctacatcgaa agttcaaaag | 1740 |
| cagcctatgc caaacaggga gagtccctgt cccccacccc cacccccaaa aaagagcaaa | 1800 |
| agcaaaccac atgatttata gacaaaagca gtgggagaga caaagaaaat acttaaacac | 1860 |
| ccactgagct gccaactagg tgtctctgac tacgggtctt ttatttgcta catatatatt | 1920 |
| tttacaaaaa tgaaatccat attgtacgct attttgctgt ctgcttcgtt cccatgtcga | 1980 |
| catgacccgc acttcttttc ccatcaataa atgtgttgtg atatttttaa aaaaaaaaaa | 2040 |
| aaaaa | 2045 |

The above mouse RISC cDNA contains a 452 amino acid open reading frame (at 30-1385). This cDNA sequence has been deposited with GenBank as Accession No. AF330052, which is hereby incorporated by reference in its entirety.

Another exemplary RISC protein or polypeptide of the present invention is a RISC protein isolated from human having an amino acid sequence corresponding to SEQ ID No: 40 as follows:

```
Met Glu Leu Ala Leu Arg Arg Ser Pro Val Pro Arg Trp Leu Leu Leu
    1 5 10 15
Leu Pro Leu Leu Leu Gly Leu Asn Ala Gly Ala Val Ile Asp Trp Pro
                          20 25 30
Thr Glu Glu Gly Lys Glu Val Trp Asp Tyr Val Thr Val Arg Lys Asp
                  35 40 45
Ala Tyr Met Phe Trp Trp Leu Tyr Tyr Ala Thr Asn Ser Cys Lys Asn
          50 55 60
Phe Ser Glu Leu Pro Leu Val Met Trp Leu Gln Gly Gly Pro Gly Gly
  65 70 75 80
Ser Ser Thr Gly Phe Gly Asn Phe Glu Glu Ile Gly Pro Leu Asp Ser
                                  85 90 95
Asp Leu Lys Pro Arg Lys Thr Thr Trp Leu Gln Ala Ala Ser Leu Leu
                        100 105 110
Phe Val Asp Asn Pro Val Gly Thr Gly Phe Ser Tyr Val Asn Gly Ser
                115 120 125
Gly Ala Tyr Ala Lys Asp Leu Ala Met Val Ala Ser Asp Met Met Val
        130 135 140
Leu Leu Lys Thr Phe Phe Ser Cys His Lys Glu Phe Gln Thr Val Pro
145 150 155 160
Phe Tyr Ile Phe Ser Glu Ser Tyr Gly Gly Lys Met Ala Ala Gly Ile
                                165 170 175
Gly Leu Glu Leu Tyr Lys Ala Ile Gln Arg Gly Thr Ile Lys Cys Asn
                        180 185 190
Phe Ala Gly Val Ala Leu Gly Asp Ser Trp Ile Ser Pro Val Asp Ser
                195 200 205
Val Leu Ser Trp Gly Pro Tyr Leu Tyr Ser Met Ser Leu Leu Glu Asp
        210 215 220
Lys Gly Leu Ala Glu Val Ser Lys Val Ala Glu Gln Val Leu Asn Ala
225 230 235 240
Val Asn Lys Gly Leu Tyr Arg Glu Ala Thr Glu Leu Trp Gly Lys Ala
                                245 250 255
Glu Met Ile Ile Glu Gln Asn Thr Asp Gly Val Asn Phe Tyr Asn Ile
                        260 265 270
Leu Thr Lys Ser Thr Pro Thr Ser Thr Met Glu Ser Ser Leu Glu Phe
                275  280  285
Thr Gln Ser His Leu Val Cys Leu Cys Gln Arg His Val Arg His Leu
        290  295  300
Gln Arg Asp Ala Leu Ser Gln Leu Met Asn Gly Pro Ile Arg Lys Lys
305  310  315  320
Leu Lys Ile Ile Pro Glu Asp Gln Ser Trp Gly Gly Gln Ala Thr Asn
                                325  330  335
Val Phe Val Asn Met Glu Glu Asp Phe Met Lys Pro Val Ile Ser Ile
                        340 345 350
Val Asp Glu Leu Leu Glu Ala Gly Ile Asn Val Thr Val Tyr Asn Gly
                355 360 365
Gln Leu Asp Leu Ile Val Asp Thr Met Gly Gln Glu Ala Trp Val Arg
        370 375 380
Lys Leu Lys Trp Pro Glu Leu Pro Lys Phe Ser Gln Leu Lys Trp Lys
385 390 395 400
Ala Leu Tyr Ser Asp Pro Lys Ser Leu Glu Thr Ser Ala Phe Val Lys
                                405 410 415
Ser Tyr Lys Asn Leu Ala Phe Tyr Trp Ile Leu Lys Ala Gly His Met
                        420 425 430
Val Pro Ser Asp Gln Gly Asp Met Ala Leu Lys Met Met Arg Leu Val
                435 440 445
Thr Gln Gln Glu
        450
```

The conserved motifs common to serine carboxypeptidases are also found in the human RISC protein sequence. The SC binding site is found at amino acids 73-82. The first catalytic domain is found at amino acids 163-170. The second catalytic domain is found at amino acids 365-373. The third catalytic domain is found at amino acids 421-437.

One nucleic acid encoding the human RISC protein or polypeptide of the present invention is a DNA molecule having a nucleotide sequence corresponding to SEQ ID No: 41 as follows:

| | |
|---|---|
| cctgttgctg atgctgccgt gcggtacttg tcatggagct ggcactgcgg cgctctcccg | 60 |
| tcccgcggtg gttgctgctg ctgccgctgc tgctgggcct gaacgcagga gctgtcattg | 120 |
| actggcccac agaggagggc aaggaagtat gggattatgt gacggtccgc aaggatgcct | 180 |
| acatgttctg gtggctctat tatgccacca actcctgcaa gaacttctca gaactgcccc | 240 |
| tggtcatgtg gcttcagggc ggtccaggcg gttctagcac tggatttgga aactttgagg | 300 |
| aaattgggcc ccttgacagt gatctcaaac cacggaaaac cacctggctc caggctgcca | 360 |
| gtctcctatt tgtggataat cccgtgggca ctgggttcag ttatgtgaat ggtagtggtg | 420 |
| cctatgccaa ggacctggct atggtggctt cagacatgat ggttctcctg aagaccttct | 480 |
| tcagttgcca caaagaattc cagacagttc cattctacat tttctcagag tcctatggag | 540 |
| gaaaaatggc agctggcatt ggtctagagc tttataaggc cattcagcga gggaccatca | 600 |
| agtgcaactt tgcgggggtt gccttgggtg attcctggat ctcccctgtt gattcggtgc | 660 |
| tctcctgggg accttacctg tacagcatgt ctcttctcga agacaaaggt ctggcagagg | 720 |
| tgtctaaggt tgcagagcaa gtactgaatg ccgtaaataa ggggctctac agagaggcca | 780 |
| cagagctgtg ggggaaagca gaaatgatca ttgaacagaa cacagatggg gtgaacttct | 840 |
| ataacatctt aactaaaagc actcccacgt ctacaatgga gtcgagtcta gaattcacac | 900 |
| agagccacct agtttgtctt tgtcagcgcc acgtgagaca cctacaacga gatgccttaa | 960 |
| gccagctcat gaatggcccc atcagaaaga agctcaaaat tattcctgag gatcaatcct | 1020 |
| ggggaggcca ggctaccaac gtctttgtga acatggagga ggacttcatg aagccagtca | 1080 |
| ttagcattgt ggacgagttg ctggaggcag ggatcaacgt gacggtgtat aatggacagc | 1140 |
| tggatctcat cgtagatacc atgggtcagg aggcctgggt gcggaaactg aagtggccag | 1200 |
| aactgcctaa attcagtcag ctgaagtgga aggccctgta cagtgaccct aaatctttgg | 1260 |
| aaacatctgc ttttgtcaag tcctacaaga accttgcttt ctactggatt ctgaaagctg | 1320 |
| gtcatatggt tccttctgac caaggggaca tggctctgaa gatgatgaga ctggtgactc | 1380 |
| agcaagaata ggatggatgg ggctggagat gagctggttt ggccttgggg cacagagctg | 1440 |
| agctgaggcc gctgaagctg taggaagcgc cattcttccc tgtatctaac tggggctgtg | 1500 |
| atcaagaagg ttctgaccag cttctgcaga ggataaaatc attgtctctg gaggcaattt | 1560 |
| ggaaattatt tctgcttctt aaaaaaacct aagatttttt aaaaaattga tttgttttga | 1620 |
| tcaaaataaa ggatgataat agatattatt ttttcttatg acagaagcaa atgatgtgat | 1680 |
| ttatagaaaa actgggaaat acaggtaccc aaagagtaaa tcaacatctg tataccccct | 1740 |
| tcccaggggt aagcactgtt accaatttag catatgtcct tgcagaattt ttttttctat | 1800 |
| atatacatat atatttttta ccaaaatgaa tcattactct atgttgtttt actatttgtt | 1860 |
| tgacatatca gtatatctga aacacctttt catgtcaata aatgttcttc tctaacatta | 1920 |
| a | 1921 |

The above human RISC cDNA contains a 452 amino acid open reading frame (nt 33-1388). This cDNA sequence has been deposited with GenBank as Accession No. NM_{—}021626, which is hereby incorporated by reference in its entirety.

Also suitable as a nucleic acid of the present invention is a nucleic acid molecule encoding a mammalian RISC protein or polypeptide that hybridizes to a complement of a nucleic acid molecule having a nucleotide sequence of either SEQ. ID. Nos. 36, 38, or 40 under stringent conditions characterized by a hybridization buffer comprising Sx SSC at a temperature of about 56° C. Alternatively, more stringent hybridization conditions may be used wherein the hybridization and/or hybridization wash buffer is 2×SSC, 1×SSC or 0.1×SSC, and the temperature is from about 56° C. to about 65° C. (including all temperatures in this range), where it is understood that “high stringency” in hybridization procedures refers generally to low salt, high temperature conditions. One skilled in the art will appreciate that conditions for nucleic acid hybridization, including temperature, salt, and the presence of organic solvents, are variable depending upon the size (i.e, number of nucleotides) and the G-C content of the nucleic acids involved, as well as the hybridization assay employed. (ee, e.g., Sambrook et al., Molecular Cloning: A Laboratory Manual, Cold Spring Laboratory, Cold Spring Harbor, N.Y. (1989); Nucleic Acid Hybridization: A Practical Approach, Haimes and Higgins, Eds., Oxford:IRL Press (1988); Hybridization with cDNA Probes User Manual, Clonetech Laboratories, CA (2000), which are hereby incorporated by reference in their entirety).

In any of the aspects described herein, the mammalian RISC protein or polypeptide may be present in an isolated form.

In one aspect of the present invention, the isolated mammalian RISC protein or polypeptide of the present invention is substantially purified. The protein or polypeptide of the present invention is preferably produced in purified form by conventional techniques. Exemplary sources for a purified protein are smooth muscle cells derived from rat, mouse, or human aorta or artery; or clonal cell lines, such as the PAC1 SMC line, which spontaneously arose from a rat pulmonary artery SMC culture (Rothman et al., “Development and Characterization Of A Cloned Rat Pulmonary Arterial Smooth Muscle Cell Line That Maintains Differentiated Properties Through Multiple Subcultures,” Circulation, 86:1977-1986 (1992), which is hereby incorporated by reference in its entirety). The PAC1 SMC cell line has several desirable attributes, including stable properties, and exhibits good transfection efficiency (Firulli et al, “A Comparative Molecular Analysis of Four Rat Smooth Muscle Cell Lines,” In Vitro Cell Dev. Biol., 34:217-226 (1998), which is hereby incorporated by reference in its entirety).

Additional sources of RISC can be derived from primary or established cell lines of transitional epithelial cells of the bladder and any cell line derived therein as well as primary or established cell lines of proximal convoluted tubular epithelial cells.

Alternatively, the protein or polypeptide of the present invention is isolated from a recombinant host cell (either eukaryotic or prokaryotic) expressing the protein or polypeptide. To isolate the protein, the host cell carrying a recombinant plasmid is propagated, homogenized, and the homogenate is centrifuged to remove bacterial debris. The supernatant is then subjected to sequential ammonium sulfate precipitation. The fraction containing the protein of the present invention is subjected to gel filtration in an appropriately sized dextran or polyacrylamide column to separate the proteins. If necessary, the protein fraction may be further purified by HPLC. Numerous methods of protein purification methods are known to those skilled in the art (Guide to Protein Purification: Methods in Enzymology, Vol 182, Deutsher and Abelson, Eds., (1997); Scope, Protein Purification: Principles and Practice, Springer-Verlag, 3rd ed., (1993); Protein Analysis and Purification Benchtop Techniques, I. Rosenberg, Birkhäuser, (1996); and Scope, “Protein Purification In The Nineties (review) Biotechnology & Applied Biochemistry 23(Part 3): 197-204 (1996), which are hereby incorporated by reference in their entirety). Therefore, purification may be carried out as described herein or using alternative methods as desired.

Mutations or variants of the above polypeptide or protein are also encompassed by the present invention.

Variants may be modified by, for example, the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of the above protein are also encompassed by the present invention. Suitable fragments can be produced by several means. In the first, subclones of the gene encoding the protein of the present invention are produced by conventional molecular genetic manipulation by subcloning gene fragments. The subclones then are expressed in vitro or in vivo in bacterial or eukaryotic cells to yield a smaller protein or peptide.

In another approach, based on knowledge of the primary structure of the protein of the present invention, fragments of the gene of the present invention may be synthesized by using the PCR technique together with specific sets of primers chosen to represent particular portions of the protein. These then would be cloned into an appropriate vector for increased expression of an accessory peptide or protein.

Chemical synthesis can also be used to make suitable fragments. Such synthesis is carried out using known amino acid sequence for a protein or polypeptide of the present invention. These fragments can then be separated by conventional procedures (e.g., chromatography, SDS-PAGE) and used in the methods of the present invention.

The present invention also relates to a nucleic acid construct having a nucleic acid molecule encoding a retinoid inducible serine carboxypeptidase protein or polypeptide. Generally, this involves inserting a nucleic acid molecule into an expression system to which the nucleic acid molecule is heterologous (i.e., not normally present), and wherein the nucleic acid molecule is operably linked to 5′ and 3′ transcriptional and translational regulatory elements (e.g. promoter, enhancer, suppressor, transcription terminator, etc.) to allow for expression of the nucleic acid molecule in a host.

In one aspect of the present invention, the nucleic acid molecule encoding a mammalian retinoid inducible serine carboxypeptidase protein or polypeptide is inserted into the expression vector in proper sense orientation and correct reading frame.

In the alternative, the nucleic acid molecule of the present invention can be inserted in the antisense (3′→5′) orientation. When a nucleic acid is inserted in the vector in the antisense orientation it is termed an “antisense” construct. Antisense nucleic acids are DNA or RNA molecules that are complementary to at least a portion of a specific mRNA molecule (Weintraub, “Antisense RNA and DNA,” Scientific American 262:40 (1990), which is hereby incorporated by reference in its entirety). Antisense methodology takes advantage of the fact that nucleic acids tend to pair with “complementary” sequences. By complementary, it is meant that polynucleotides are capable of base-pairing according to the standard Watson-Crick rules. In the target cell, the antisense nucleic acids hybridize to a target nucleic acid and interfere with transcription, and/or RNA processing, transport, translation, and/or stability. The overall effect of such interference with the target nucleic acid function is the disruption of protein expression. Antisense RNA constructs, or DNA encoding such a construct, employing the RISC-encoding nucleic acids of the present invention, may be used to inhibit gene transcription or translation, or both, within a host cell, either in vitro or in vivo, such as within a mammalian host, even a human subject. The description herein of the preparation of nucleic acid constructs, expression systems, and host cells applies to both sense and the antisense constructs using the nucleic acids of the present invention.

The introduction of a gene into a host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements, and which is capable of transferring gene sequences between cells. Thus, the term includes cloning and expression vectors, as well as viral vectors, including adenoviral, retroviral vectors, and lentiviral vectors.

Exemplary vectors include, without limitation, the following: lambda vector system gt11, gt WES.TB, Charon 4, and plasmid vectors such as pBR322, pBR325, pACYC177, pACYC184, pUC8, pUC9, pUC18, pUC19, pLG339, pR290, pKC37, pKC101, SV 40, pbluescript II SK+/− or KS+/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof.

The nucleic acid molecules of the present invention may be inserted into any of the many available expression vectors and cell systems using reagents that are well known in the art. U.S. Pat. No. 4,237,224 to Cohen and Boyer, which is hereby incorporated by reference in its entirety, describes the production of expression systems in the form of recombinant plasmid vectors using restriction enzyme cleavage and ligation with DNA ligase.

A variety of host-vector systems may be utilized to express the protein-encoding sequence of the present invention. Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, retrovirus, lentivirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Transcription of DNA is dependent upon the presence of a promoter, which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in, or may not function in, a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzmology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the 17 phage promoter, lac promotor, trp promotor, recA promotor, ribosomal RNA promotor, the PR and PL promoters of coliphage lambda and others, including but not limited, to lacUV5, ompF, bla, Ipp, and the like, may be used to direct high levels of transcription of adjacent DNA segments. Additionally, a hybrid trp-lacUV5 (tac) promotor or other E. coli promoters produced by recombinant DNA or other synthetic DNA techniques may be used to provide for transcription of the inserted gene.

Common promoters suitable directing expression in mammalian cells include, without limitation, SV40, MMTV, metallothionein-1, adenovirus E1a, CMV, immediate early, immunoglobulin heavy chain promoter and enhancer, and RSV-LTR.

Bacterial host cell strains and expression vectors may be chosen which inhibit the action of the promotor unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The nucleic acid expression vector, which contains a promotor, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant nucleic acid or other techniques involving incorporation of synthetic nucleotides may be used.

The nucleic acid molecule of the present invention, appropriate transcriptional and translational regulatory elements, as described above, and any additional desired components, including, without limitation, enhancers, leader sequences, markers, etc., are cloned into the vector of choice using standard cloning procedures in the art, such as described in Sambrook et al., Molecular Cloning: A Laboratory Manual, Cold Spring Laboratory, Cold Spring Harbor, N.Y. (1989), Ausubel et al., “Short Protocols in Molecular Biology,” New York:Wiley (1999), and U.S. Pat. No. 4,237,224 to Cohen and Boyer, which are hereby incorporated by reference in their entirety.

Once the nucleic acid construct containing the nucleic acid molecule of the present invention has been cloned into an expression system, it is ready to be incorporated into a host cell by means of transformation and replicated in unicellular cultures including prokaryotic organisms and eukaryotic cells grown in tissue culture. Accordingly, another aspect of the present invention relates to a method of making a recombinant cell having a nucleic construct including a nucleic acid molecule encoding a RISC protein or polypeptide of the present invention. Basically, this method is carried out by transforming a host cell with the vector containing the nucleic acid construct of the present invention under conditions effective to yield transcription of the nucleic acid molecule in the host cell. Preferably, the nucleic acid construct of the present invention is stably inserted into the genome of the recombinant host cell as a result of the transformation.

Such incorporation can be carried out by various forms of transformation, depending upon the vector/host cell system.

Recombinant molecules can be introduced into cells via transformation, particularly transduction, conjugation, mobilization, or electroporation. The nucleic acid sequences are cloned into the host cell using standard cloning procedures known in the art, as described by Sambrook et al., Molecular Cloning: A Laboratory Manual, Second Edition, Cold Spring Laboratory, Cold Spring Harbor, N.Y. (1989), which is hereby incorporated by reference in its entirety. Suitable host cells include, but are not limited to, bacteria, virus, yeast, plant, mammalian cells, and the like.

Transient expression in protoplasts allows quantitative studies of gene expression since the population of cells is very high (on the order of 10⁶). To deliver nucleic acid inside protoplasts, several methodologies have been proposed, but the most common are electroporation (Neumann et al., “Gene Transfer into Mouse Lyoma Cells by Electroporation in High Electric Fields,” EMBO J. 1: 841-45 (1982); Wong et al., “Electric Field Mediated Gene Transfer,” Biochem. Biophys. Res. Commun. 30;107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81: 7161-65 (1984), which are hereby incorporated by reference in their entirety) and polyethylene glycol (PEG) mediated DNA uptake, Sambrook et al., Molecular Cloning: A Laboratory Manual, Chap. 16, 2d Edition, Cold Spring Laboratory, Cold Spring Harbor, N.Y. (1989), which is hereby incorporated by reference in its entirety). During electroporation, the nucleic acid is introduced into the cell by means of a reversible change in the permeability of the cell membrane due to exposure to an electric field. PEG transformation introduces the nucleic acid by changing the elasticity of the membranes. Unlike electroporation, PEG transformation does not require any special equipment. Another appropriate method of introducing the gene construct of the present invention into a host cell is fusion of protoplasts with other entities, either minicells, cells, lysosomes, or other fusible lipid-surfaced bodies that contain the chimeric gene. Fraley, et al., Proc. Natl. Acad. Sci. USA, 79:1859-63 (1982), which is hereby incorporated by reference in its entirety.

Stable transformants are preferable for the methods of the present invention, which can be achieved by using variations of the methods above as described in Sambrook et al., Molecular Cloning: A Laboratorv Manual, Chap. 16, Second Edition, Cold Spring Laboratory, Cold Spring Harbor, N.Y. (1989), which is hereby incorporated by reference in its entirety.

The host cell transformed with the nucleic acid construct of the present invention having a nucleic acid encoding a mammalian RISC protein or polypeptide of the present invention can be in vivo or in vitro.

The nucleic acid molecules of the present invention can also be used to detect the presence, absence, or changes in the progression or regression of a vascular disease or disorders in a subject. Diseases or disorders that may be detected by the gene amplification method of the present invention include, but are not limited to, vascular hyperplasia, atherosclerosis, restenosis, glomerulonephritides, hypertension, obstructive bladder disease, tubulosclerosis, asthma, or interstitial tubular disease.

Another aspect of the present invention relates to a method of detecting the presence, absence, or changes in the progression or regression of a vascular disease or disorder in a subject. This method involves providing a tissue or body fluid sample from a subject, and contacting the sample with a nucleic acid molecule encoding a RISC protein or polypeptide of the present invention, or a fragment thereof, in a gene amplification reaction. The nucleic acid that is used in the contacting step may be either a primer or a probe, having a nucleotide sequence that is sufficiently homologous to initiate an amplification reaction by hybridization to a target nucleic acid substrate in the sample. As used herein, a “primer” and a “probe” are similar in the requirement that each is suitable for hybridizing to a portion of the target nucleic acid in the sample under appropriate conditions. However, the use of a primer is generally indicative of a reaction in which a polymerase is added to the reaction to allow for geometric or logarithmic amplification of the target. In the case of a linear amplification-detection assay, the nucleic acid is referred to as a probe. In either case, suitable nucleic acids for this aspect of the present invention include, without limitation, DNA and RNA, including an mRNA molecule, for a mammalian retinoid inducible serine carboxypeptidase protein or polypeptide of the present invention.

In this aspect of the present invention the detection procedure may be a polymerase chain reaction (“PCR”); RT-PCR; in situ PCR, oligonucleotide ligation assay (OLA), ligase amplification reaction (LAR); ligation chain reaction (LCR), or any other detection assay that is capable of deterimining that amplification of the target nucleic acid has or has not occurred, thereby indicating the presence, absence, or a change in the progression or regression of a vascular disease or disorder in the subject. Detection of an amplification product may indicate the presence of a vascular disease or disorder in the subject, while the absence of an amplification product may indicate the absence thereof, and any change in the extent to which amplification products form (i.e., over time) may indicate a change in the progression or regression thereof. Such methods and the conditions therefor are known to those in the art or as described in the literature, such as in Sambrook et al., Molecular Cloning: A Laboratory Manual, Second Edition, Cold Spring Laboratory, Cold Spring Harbor, N.Y. (1989), and Barany, F. “LAR Using a Thermal Stable DNA Ligase,” Proc. Natl. Acad. Sci. USA, 88:189 (1991), which are hereby incorporated by reference in their entirety.

Exemplary nucleic acid molecules suitable as primers in the present invention include, without limitation, the nucleotide sequences shown in Table 1 below.

TABLE 1
Forward and Reverse Probes/Primers for Use
in Hybridization or Amplification Procedures

| Exont⁵⁵⁴ | Forward | | Reverse | |
|---|---|---|---|---|
| 1 | CCTGTTGCTGATGCTGCC | (SEQ ID No: 42) | CTGCGTTCAGGCCCAGCA | (SEQ ID No: 43) |
| 2 | GAGCTGTCATTGACTGGC | (SEQ ID No: 44) | CTGAAGCCACATGACCAG | (SEQ ID No: 45) |
| 3 | GGCGGTCCAGGCGGTTCT | (SEQ ID No: 46) | CCAGGTGGTTTTCCGTGG | (SEQ ID No: 47) |
| 4 | CTCCAGGCTGCCAGTCTC | (SEQ ID No: 48) | CTGGAATTCTTTGTGGCA | (SEQ ID No: 49) |
| 5 | ACAGTTCCATTCTACATT | (SEQ ID No: 50) | CTTATAAAGCTCTAGACC | (SEQ ID No: 51) |
| 6 | GCCATTCAGCGAGGGACC | (SEQ ID No: 52) | CAACAGGGGAGATCCAGG | (SEQ ID No: 53) |
| 8 | CGAAGACAAAGGTCTGGC | (SEQ ID No: 54) | TTCTGCTTTCCCCCACAG | (SEQ ID No: 55) |
| 9 | AACACAGATGGGGTGAACTT | (SEQ ID No: 56) | CTAGGTGGCTCTGTGTGA | (SEQ ID No: 57) |
| 10 | TTTGTCTTTGTCAGCGCCAC | (SEQ ID No: 58) | GGCCTCCCCAGGATTGAT | (SEQ ID No: 59) |
| 11 | CCAGGCTACCAACGTCTT | (SEQ ID No: 60) | GACCCATGGTATCTACGA | (SEQ ID No: 61) |
| 12 | CCTGGGTGCGGAAACTGAA | (SEQ ID No: 62) | GACCAGCTTTCAGAATCCA | (SEQ ID No: 63) |
| 13 | CTTCTGACCAAGGGGAGA | (SEQ ID No: 64) | GTTAGAGAAGAACATTTATTGACAT | (SEQ ID No: 65) |

⁵⁵⁴ Refers to human RISC exon sequence derives from

The present invention also relates to a method of detecting the presence, absence, or changes in the progression or regression of a vascular disease or disorder in which a tissue or body fluid sample is provided by a subject, and that sample is contacted with a nucleic acid probe under conditions effective to cause the probe and any target nucleic acid present in the sample to form a hybridization complex. Suitable as a nucleic acid probe in this aspect of the present invention is a nucleic acid molecule, or a fragment thereof, that encodes a RISC protein or polypeptide of the present invention, or is complementary thereto. Such a nucleic acid probe may be selected from the group consisting of DNA and RNA, including an mRNA molecule for a retinoid inducible serine carboxypeptidase protein or polypeptide of the present invention. Exemplary probes are those listed in Table 1 above.

Determination of the formation of any hybridization complex in this aspect of the present invention may be carried out by Northern blot (Thomas, P. S., “Hybridization of Denatured RNA and Small DNA Fragments Transferred to Nitrocellulose,” Proc. Nat'l. Acad. Sci. USA, 77:5201-05 (1980), which is hereby incorporated by reference in its entirety), Southern blot (Southern, E. M., “Detection of Specific Sequences Among DNA Fragments Separated by Gel Electrophoresis,” J. Mol. Biol., 98:503-17 (1975), which is hereby incorporated by reference in its entirety), PCR (Erlich, H. A., et. al., “Recent Advances in the Polymerase Chain Reaction”, Science 252:1643-51 (1991), which is incorporated herein by reference in its entirety), in-situ hybridization (Nucleic Acid Hybridization: A Practical Approach, Haimes and Higgins, Eds., Oxford:IRL Press (1988), which is hereby incorporated by reference in its entirety), in-situ PCR (Haase et al., “Amplification and Detection of Lentiviral DNA Inside Cells,” Proc. Natl. Acad. Sci. USA, 87(13):4971-5 (1991), which is hereby incorporated by reference in its entirety), or other suitable hybridization assays known in the art. Nucleic acid probe are generally “tagged” using either traditional radioactive labeling and detection methods (Sambrook et al., Molecular Cloning: A Laboratory Manual, Cold Spring Laboratory, Cold Spring Harbor, N.Y. (1989), which is hereby incorporated by reference in its entirety), or with non-radioactive materials, such as biotin, digoxigenin, various fluorochromes, or haptens (Hybridization with cDNA Probes User Manual, Clonetech Laboratories, CA (2000); Harvey, et al., Protocols for Nucleic Acid Analysis by Nonradioactive Probes, Ed. P. G. Isaac, Humana Press, New Jersey, pp. 93-100, 1994, which are hereby incorporated by reference in their entirety). The labeling method and assay conditions will be dictated by the choice of assay system to be employed. Those methods most suitable are detection assays in which the contacted fluid sample or tissue is washed to remove any probe not bound (i.e., not hybridized to target). Detection of a hybridization complex may indicate the presence of a vascular disease or disorder in the subject, while the absence of a hybridization complex may indicate the absence thereof, and any change in the extent to which hybridization complexes form (i.e., over time) may indicate a change in the progression or regression thereof.

Another aspect of the present invention relates to an isolated antibody, or binding portion thereof, that binds to a mammalian RISC protein or polypeptide.

Examples of suitable antigens for producing the antibody or binding portion thereof of the present invention include, without limitation, the rat RISC protein or polypeptide having an amino acid corresponding to SEQ ID No: 36; the mouse RISC protein or polypeptide having an amino acid corresponding to SEQ ID No: 38; and the human RISC protein or polypeptide having an amino acid corresponding to SEQ ID No: 40.

Also suitable as antigens for producing the antibody of the present invention are specific domains of any RISC protein or polypeptide. These domains include, without limitation, the serine carboxypeptidase substrate binding domains described above and the first, second, and third catalytic domains described above (see FIG. 1).

Antibodies of the present invention include those which are capable of binding to a protein or polypeptide of the present invention and inhibiting the activity of such a polypeptide or protein, (i.e., a neutralizing antibody). The disclosed antibodies may be monoclonal or polyclonal.

Monoclonal antibody production may be effected by techniques which are well-known in the art. Monoclonal Antibodies—Production, Engineering and Clinical Applications, Ritter et al., Eds. Cambridge University Press, Cambridge, UK (1995), which is hereby incorporated by reference in its entirety. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature, 256:495 (1975), which is hereby incorporated by reference in its entirety.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (PEG) or other fusing agents. Milstein and Kohler, Eur. J. Immunol., 6:511 (1976), which is hereby incorporated by reference in its entirety. This immortal cell line, which is preferably murine, but may also be derived from cells of other mammalian species, including without limitation, rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled approximately every two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in Harlow, et. al., Eds., Antibodies: A Laboratory Manual, Cold Spring Harbor Laboratory, New York (1988), which is hereby incorporated by reference in its entirety.

It is also possible to use the anti-idiotype technology to produce monoclonal antibodies that mimic an epitope. As used in this invention, “epitope” means any antigenic determinant on an antigen to which the paratope of an antibody binds. Epitopic determinants usually consist of chemically active surface groupings of molecules, such as amino acids or sugar side chains and usually have specific three dimensional structural characteristics, as well as specific charge characteristics. For example, an anti-idiotype monoclonal antibody made to a first monoclonal antibody will have a binding domain in the hypervariable region that is the image of the epitope bound by the first monoclonal antibody.

In addition to whole antibodies, the present invention encompasses binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, (pp. 98-118) Academic Press: New York (1983), and Harlow et al., Antibodies: A Laboratory Manual, Cold Spring Harbor Laboratory, New York (1988), which are hereby incorporated by reference in their entirety, or other methods known in the art.

The present invention also relates to a method of detecting the presence, absence, or changes in the progression or regression of a vascular disease or disorder that involves contacting a tissue or fluid sample from a subject with an antibody or binding portion thereof, under conditions effective to permit formation of an antigen-antibody/binding portion complex. The formation of an antigen-antibody/binding portion complex is determined by using an assay system. Detection of antigen-antibody/binding portion complex may indicate the presence of a vascular disease or disorder, while the absence of antigen-antibody/binding portion complex may indicate the absence thereof, and any change in the extent to which antigen-antibody/binding portion complex forms (i.e., over time) may indicate a change in the progression or regression thereof. Exemplary diseases or disorders which can be detected are listed previously herein.

Antibodies or binding portions thereof suitable for this aspect of the present invention include those which bind to a mammalian RISC protein or polypeptide.

Examples of an assay system suitable for the determination of a RISC an antigen-antibody/binding portion complex include, without limitation, an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay. Conditions suitable for formation of the antigen-antibody/binding portion complex will dictated by the choice of assay system, and are known or can be readily determined by those skilled in the art.

The present invention also relates to a method of inhibiting smooth muscle cell growth. This method involves increasing the intracellular concentration of a retinoid-inducible protein or polypeptide in a smooth muscle cell under conditions effective to inhibit growth of the smooth muscle cell.

Exemplary retinoid-inducible proteins or polypeptides suitable for this method of the present invention include retinoid inducible serine carboxypeptidase (RISC), spermidine/spermine N-acetyltransferase (SSAT) (GenBank Accession No. L10244, which is hereby incorporated by reference in its entirety), src suppressed C kinase substrate (SSeCKS) (GenBank Accession No. U23146, which is hereby incorporated by reference in its entirety), epithelin (GenBank Accession No. X62322, which is hereby incorporated by reference in its entirety), a8-integrin (GenBank Accession No. AF148797, which is hereby incorporated by reference in its entirety), vascular cell adhesion molecule-1 (VCAM-1) (GenBank Accession No. M84488, which is hereby incorporated by reference in its entirety), tissue transglutaminase (tTG) (GenBank Accession No. AF106325, which is hereby incorporated by reference in its entirety), lactate dehydrogenase-B (LDH-B) (GenBank Accession No. U07181, which is hereby incorporated by reference in its entirety), lectin-like oxidized LDL receptor (LOX-1) (GenBank Accession No. AB005900, which is hereby incorporated by reference in its entirety), retinol dehydrogenase (RDH) (GenBank Accession No. AF061743, which is hereby incorporated by reference in its entirety), cathepsin-L (GenBank Accession No. S85184, which is hereby incorporated by reference in its entirety), ceruloplasmin (GenBank Accession No. L33869, which is hereby incorporated by reference in its entirety), importin a (GenBank Accession No. AJ130946, which is hereby incorporated by reference in its entirety), endolyn (GenBank Accession No. AJ238574, which is hereby incorporated by reference in its entirety), stoned B/TFIIAα/β-like factor (SALF) (GenBank Accession No. AF026169, which is hereby incorporated by reference in its entirety), or a combination thereof. As described in the Examples below, a modified suppression subtractive hybridization assay was performed to uncover these genes induced by atRA in cultured SMCs. Northern blotting studies confined the induction of these genes, many of which have heretofore been unrecognized as retinoid-inducible. Temporal expression and CHX studies allowed the categorization of these genes as either immediate-early (LOX-1, endolyn, Stoned B/TFIIAα/β-like factor, Src Suppressed C Kinase Substrate, and tissue transglutaminase) or delayed (cathepsin-L, ceruloplasmin, epithelin, importin α, α₈-integrin, lactate dehydrogenase B, retinol dehydrogenase, spermidine/spermine N¹-acetyltransferase, and VCAM-1) retinoid-response genes. A survey of rat tissues showed two of the genes (tissue transglutaminase and α₈-integrin) to be highly restricted to vascular tissue. In situ hybridization verified expression of both tissue transglutaminase and α₈-integrin to SMC in balloon-injured rat carotid artery. These findings unveiled a new retinoid-response gene set that may be useful to define molecular pathways involved in the antagonistic effects of retinoids on SMC growth and neointimal formation, and are highly suitable as retinoid inducible proteins or polypeptides in the present invention.

The retinoid inducible proteins or polypeptides can be specific for the type of smooth muscle cell whose growth is to be inhibited. Thus, human retinoid inducible proteins or polypeptides can be used to suppress human smooth muscle cell growth, rat retinoid inducible proteins or polypeptides can be used to suppress rat smooth muscle cell growth, and mouse retinoid inducible proteins or polypeptides can be used to suppress mouse smooth muscle cell growth, etc.

In one aspect of the present invention, increasing the intracellular concentration of a retinoid-inducible protein or polypeptide in a smooth muscle cell involves introducing the retinoid-inducible protein or polypeptide into the smooth muscle cell. This may be carried out by contacting the smooth muscle cell with a delivery vehicle containing a retinoid-inducible protein or polypeptide. An exemplary delivery vehicle for this method is a liposome vehicle containing the retinoid-inducible protein or polypeptide.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Wang et al., “pH-Sensitive Immunoliposomes Mediate Target-Cell-Specific Delivery and Controlled Expression of a Foreign Gene in Mouse,” Proc. Natl. Acad. Sci. USA, 84(22):7851-7855 (1987); Biochemistry 28:908 (1989), which are hereby incorporated by reference in their entirety). When liposomes are endocytosed by a target cell, for example, they can be routed to acidic endosomes which will destabilize the liposome and result in drug release.

The liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

Alternatively, the delivery vehicle for the retinoid inducible protein or polypeptide includes an enzymatically stable conjugate that includes a polymer. The retinoid-inducible protein or polypeptide is chemically conjugated to the polymer.

Other suitable protein delivery systems may be used, including, without limitation, a transdermal patch, and implantable or injectable protein depot compositions, which provide long-term delivery of fusion proteins (U.S. Pat. No. 6,331,311 to Brodbeck et al., which is hereby incorporated by reference in its entirety). Other delivery systems which are known to those of skill in the art can also be employed to achieve the desired delivery of the fusion protein to smooth muscle cells in vivo to effect this aspect of the present invention.

In another aspect of the present invention, the intracellular concentration of a retinoid-inducible protein or polypeptide in smooth muscle cells is increased by transforming the smooth muscle cell with a nucleic acid encoding the retinoid-inducible protein or polypeptide of the present invention under conditions effective for expression of the retinoid-inducible protein or polypeptide in the transformed smooth muscle cell.

This aspect can be carried out by transforming the smooth muscle cell with an infective transformation vector harboring the nucleic acid encoding the retinoid-inducible protein or polypeptide. Exemplary infective transformation vectors include, without limitation, an adenovirus vector, a retrovirus vector, or a lentivirus vector harboring the nucleic acid encoding the retinoid-inducible protein or polypeptide. Such vectors, prepared as described above with suitable transcriptional and translational regulatory elements, are capable of expressing the retinoid inducible protein or polypeptide in the transformed SMC. In accordance with this aspect of the present invention, the transformed SMC is preferably within a mammalian organism (i.e., effectively providing gene therapy).

The present invention also relates to a method of treating vascular hyperplasia in which the intracellular concentration of a retinoid-inducible protein or polypeptide is increased in vascular smooth muscle cells at a site of vascular hyperplasia, under conditions effective to treat the vascular hyperplasia. This involves the introduction of a retinoid-inducible protein or polypeptide into one or more smooth muscle cells at the site of vascular hyperplasia.

Exemplary retinoid-inducible proteins or polypeptides for use in this aspect of the present invention include, without limitation, those describe above.

The introduction of a retinoid-inducible protein or polypeptide of the present invention into one or more smooth muscle cells at the site of vascular hyperplasia may be carried out by employing a delivery vehicle having the retinoid-inducible protein or polypeptide. Exemplary delivery vehicles for this aspect of the present invention include, without limitation, a fusion protein having a retinoid-inducible protein or polypeptide of choice and a ligand domain recognized by smooth muscle cells; a liposome vehicle, in its various forms as described above and known in the art; or an enzymatically stable conjugate having a polymer and the retinoid-inducible protein or polypeptide conjugated to the polymer. Other delivery vehicles known to those in the art are also suitable, including a catheter device to deploy the delivery vehicles as described above.

This method also encompasses effecting an increase in the intracellular concentration of a retinoid-inducible protein or polypeptide in SMCs at the site of hyperplasia by transforming one or more smooth muscle cells with a nucleic acid encoding the retinoid-inducible protein or polypeptide under conditions effective for expression of the retinoid-inducible protein or polypeptide in the transformed one or more smooth muscle cells. This aspect can be carried out by transforming the smooth muscle cell with an infective transformation vector harboring the nucleic acid encoding the retinoid-inducible protein or polypeptide. Exemplary infective transformation vectors include, without limitation, an adenovirus vector, a retrovirus vector, or a lentivirus vector harboring the nucleic acid encoding the retinoid-inducible protein or polypeptide. Such vectors, prepared as described above with suitable transcriptional and translational regulatory elements, are capable of expressing the retinoid inducible protein or polypeptide in the transformed SMC.

The present invention also relates to a method of inhibiting the activity of extracellular regulated kinase (ERK). This involves contacting ERK with a retinoid-inducible protein or polypeptide under conditions effective to inhibit the activity of ERK. This method of contacting inhibits the phosphorylation of ERK. Phospho-ERK represents a nodal point of signal transduction to the interior of the cell, including the nucleus where changes in gene transcription are known to be mediated by phospho-ERK (Kyriakis and Avruch, “Mammalian Mitogen-Activated Protein Kinase Signal Transduction Pathways Activated by Stress and Inflammation,” Physiological Reviews 81:807-869 (2001), which is hereby incorporated by reference in its entirety). Without being bound by theory, it is believed that RISC is likely modulating growth-related signals by inhibiting receptor-ligand interactions (through a proteolytic event).

The present invention also relates to a non-human transgenic animal which contains a functional transgene encoding a functional retinoid inducible protein or polypeptide, or variants thereof. Transgenic animals expressing retinoid inducible transgenes, recombinant cells lines derived from such animals, and transgenic embryos may be useful in methods for screening and identifying agents that induce or suppress function of retinoid inducible genes. The transgenic animal is produced by integration of the transgene into the genome in a manner that permits the expression of the transgene. Methods for producing transgenic animals are generally described in U.S. Pat. No. 4,873,191 to Wagner et al; Brinster et al. (1985); Manipulating the Mouse Embryo: A Laboratory Manual, 2d edition, Hogan et al., eds., Cold Spring Harbor Laboratory Press (1994), which are hereby incorporated by reference in their entirety.

As another aspect of the invention, knowledge of the nucleic acid sequences for the retinoid inducible proteins or polypeptides of the present invention enables one skilled in the art to generate knockout animal strains that lack functional copies of the genes. Thus, the present invention also relates to non-human transgenic animals whose somatic and germ cells lack, or possess a disruption in, a gene encoding a RISC protein or polypeptide involved the regulation of a negative growth response in retinoid-treated SMCs. Alternatively, a non-human transgenic animal can be generated whose SMCs alone contain a disruption in a retinoid inducible gene as described herein. Neointimal formation is a complex process involving several cell types and myriad signaling cues that cannot be adequately modeled in vitro. Therefore, the knockout animal strains of the present invention will provide excellent model systems for studying the role of retinoid-inducible proteins in normal and pathological vessel wall growth responses as well as other diseases or disorders of the type identified above; the role of all-trans retinoic acid in blocking neointimal formation following mechanical injury; and the effects of retinoid treatment in vascular disease state Harmon et al., “Strain-Dependent Vascular Remodeling Phenotypes in Inbred Mice,” Am. J. Pathol., 156:1741-1748 (2000); Miano and Berk, “Retinoids: Versatile Biological Response Modifiers of Vascular Smooth Muscle Phenotype,” Circ Res. 87:355-362 (2000); Sate et al., “A Mouse Model of Vascular Injury That Induces Rapid Onset of Medial Cell Apoptosis Followed by Reproducible Neointimal Formation, J. Mol. Cell Cardio., 32:2097-2104 (2000), which are hereby incorporated by reference in their entirety).

Suitable knock-out animals include mice, rats, and any other non-human animal model for vascular disease in which the loss of a functional retinoid-inducible gene in the animal (or, at a minimum, in SMCs of the animal) would be useful for studying how homologous genes behave as negative growth regulators and tumor suppressors in humans.

A retinoid-inducible gene knock-out animal will be characterized by a down-regulation in (or complete lack of) the antiproliferative response in SMC of the absent retinoid-inducible protein or polypeptide in response to all-trans-retinoic acid, leading to a phenotype characterized by an increase in SMC growth and a susceptibility to hyperplasia, neointimal formation, and other vascular disorders. This phenotype is conferred to the animal by disruption of expression of the retinoid-inducible nucleic acid eliminated in the preparation of the knock-out vector. This disruption occurs as a result of meiotic homologous recombination between a replacement vector nucleic acid sequence and the host animal genes. Homologous recombination is carried out essentially according to the method of Gan et al., “POU Domain Factor Bm-3b Is Required For the Development Of a Large Set Of Retinal Ganglion Cells,” Proc. Natl. Acad. Sci. USA, 93:3920-3925-92 (1996), which is incorporated herein by reference in its entirety. It is understood that the replacement vector nucleic acid sequence can comprise any known nucleic acid sequence (i.e., DNA sequence) provided that it disrupts the natural retinoid-inducible animal gene upon homologous recombination in a manner sufficient to prevent expression of the chosen retinoid-inducible protein or polypeptide.

Briefly, a targeting vector containing the desired mutation is introduced into embryonic-derived stem (ES) cells by electroporation, microinjection or other like means. In some of the ES cells, the targeting vector pairs with the cognate chromosomal DNA sequence and transfers the mutation to the genome by homologous recombination. Screening procedures, enrichment procedures, or hybridization procedures are then utilized to identify those transformed ES cells in which the targeted event has occurred. An appropriate cell is then cloned and maintained as a pure population. The transformed ES cells are injected into blastocysts of a preimplantation embryo and the blastocyst is surgically transferred to the uterus of a foster mother, where development is allowed to progress to term. Chimeric offspring heterozygous for the desired trait are then mated to obtain homozygous individuals, and colonies characterized by deficiency in the targeted gene are established.

In accordance with the present invention, the animal's RISC gene is disrupted (i.e., chromosomal defect introduced into the respective gene locus) using a vector. Examples of such vectors include, without limitation, (1) an insertion vector as described by Gan et al., “POU Domain Factor Bm-3b Is Required For the Development Of a Large Set Of Retinal Ganglion Cells,” Proc. Natl. Acad. Sci. USA, 93:3920-3925-92 (1996); which is hereby incorporated by reference in its entirety; (2) a vector based upon promoter trap, polyadenylation trap, “hit and run” or “tag-and-exchange” strategies, as described by Bradley, A., et al., “Modifying the Mouse: Design and Desire,” Biotechnology 10:534-39 (1992); and Askew, R., et al., “Site-Directed Point Mutations in Embryonic Stem Cells: a Gene Targeting Tag-and-Exchange Strategy,” Mol. Cell Biol., 13:4115-24 (1993), both of which are incorporated herein by reference in their entirety. These vectors may or may not include negative selection markers, which when used, may allow enhancement of targeted recombinant isolation. Mansour, S. L., et al., “Disruption of the Proto-Oncogene int-2 in Mouse Embryo-Derived Stern Cells: a General Strategy for Targeting Mutations to Non-Selectable Genes,” Nature, 336:348-52 (1988); and McCarrick, J. W., et al., “Positive-Negative Selection Gene Targeting with the Diphtheria Toxin A-chain Gene in Mouse Embryonic Stem Cells,” Transgen. Res., 2:183-90 (1993), both of which are incorporated herein by reference in their entirety. These markers may be part of the targeting vector or may be co-transfected into the ES cells.

In producing a knock-out animal according to the present invention, transformed cell lines deficient for a RISC gene of the present invention can be identified by standard techniques in the art. Once identified, these host cells are cultured under conditions which facilitate growth of the cells as will be apparent to one skilled in the art. Thereafter, stable transformants may be selected on the basis of the expression of one or more appropriate gene markers present or inserted into the replacement vector. The expression of the marker genes should indicate the targeted or desired disruption of the target gene. It is understood that any known gene marker may be used herein. Such gene markers can be derived from cloning vectors, which usually contain a positive marker gene.

In order to understand the role of a RISC gene product in SMCs, a cell-specific knock-out of the RISC gene can be produced using a gene inactivation targeting system. An exemplary gene inactivation targeting system of the present invention is a Cre-lox knock-out of the RISC gene. Such a system is based on the use of the site-specific recombinase (“Cre”) that catalyzes recombination between two 34 bp loxP recognition sites. Gene inactivation is accomplished by flanking (“floxing”) the RISC locus with two loxP sites using a homologous recombination technique, and then “delivering” Cre to excise the intervening DNA including the exon from the chromosome, thereby generating a “null” allele in all cells where Cre is active. “Delivery” of Cre in a transgenic mouse, for example, can be achieved by crossing mice carrying the “floxed” RISC locus with transgenic Cre-expressing mice (Feil et al., “Ligand-activated Site-Specific Recombination in Mice,” Proc Natl Acad Sci USA, 93:10887-10890 (1996); Metzger et al., “Engineering the Mouse Genome by Site-Specific Recombination in Mice,” Curr Opin Biotechnol 10:470476 (1999), which are hereby incorporated by reference in their entirety). The knock-out is made SMC-specific by utilizing, e.g., the smooth muscle cell promoter SM22a in this Cre-lox plasmid of the gene inactivation system (KUhbander et al., “Temporally Controlled Somatic Mutagenesis in Smooth Muscle Cell,” Genesis 28:15-22 (2000), which is hereby incorporated by reference in its entirety). The functional role of any RISC gene product can be determined by assessing the vessel cell wall growth under normal and pathological conditions using a Cre-lox gene targeting knock-out with the desired gene, or portion thereof, floxed as described herein. This system is described in greater detail in Example 3, infra.

Exemplary nucleic acids for preparation of the knock-out transgenic animal of the present invention are any that encode a retinoid-inducible protein or polypeptide, including, without limitation, those corresponding to SEQ. ID. Nos. 36 or 38.

### EXAMPLES

### Example 1

### Identification of A Set of Retinoid Response Genes in Cultured SMC

Cell Culture

Cultured rat aortic SMC (RASMC) were derived from aortas of male Sprague-Dawley rats by a combined explant-enzymatic digestion procedure as described previously (Miano et al., “Expression of the Smooth Muscle Cell Calponin Gene Marks the Early Cardiac and Smooth Muscle Cell Lineages During Mouse Embryogenesis,” J. Biol. Chem. 271:7095-7103 (1996), which is hereby incorporated by reference in its entirety). These cells were grown in Dulbecco's Modified Eagle's Medium (DMEM) supplemented with 10% fetal bovine serum (FBS), 2 mM L-glutamine and antibiotics. Human coronary artery SMC (HCASMC) were obtained from a commercial vendor and grown in SmGM-2 media (Clonetics, San Diego, Calif.). Experiments were performed with cultured COS-7, PAC-1 SMC, and RASMC between passages 10 and 20, or hCASMC between passage 5 and 10. Unless indicated otherwise, cells were grown on 100-mm dishes and treated with either 2×10⁻mol/L all-trans-retinoic acid (atRA) or an equal volume of dimethylsulfoxide (DMSO) for various times when culture was 70-80% confluent.

Suppression Subtractive Hybridization and Differential Screening

Suppression subtractive hybridization (SSH) was performed using a PCR-Select™ cDNA Subtraction Kit according to the manufacturer's instructions (CLONTECH Laboratories, Inc., Palo Alto, Calif.). Two time points (12 and 72 hr) of total RNA (2 μg/time point) were pooled from RASMC treated with either 2×10⁻⁶ mol/L atRA (tester” cDNA pool) or an equal volume of DMSO (“driver” cDNA pool). Hybridizing excess driver cDNA to the tester cDNA pool resulted in a subtracted library of clones containing atRA-induced transcripts. Following transformation in TOP10F E. coli, individual clones were randomly selected for differential screening using the PCR-Select™ Differential Screening Kit (CLONTECH Laboratories, Inc., Palo Alto, Calif.). Briefly, cloned inserts were PCR-amplified using nested primers as specified by the manufacturer. PCR products were then arrayed in duplicate on 48-well slot-blot nylon membranes. One membrane was hybridized to radiolabeled tester cDNA and the other to radiolabeled driver cDNA. A third membrane was used to back-hybridize cloned cDNAs in order to reduce the number of duplicates. Positively identified clones were sequenced on both strands (University of Rochester Core Nucleic Acid Laboratory) and analyzed with the Genetics Computer Group's (GCG) suite of software programs (Version 10.1, Madison Wis.).

RNA Isolation and Northern Blotting

Total RNA from cultured RASMC and rat tissues was isolated by the acid phenol-guanidinium isothiocyanate method (Chomczynski et al., “Single-Step Method of RNA Isolation by Acid Guanidinium Thiocyanate-Phenol-Chloroform Extraction,” Anal. Biochem. 162:156-159 (1987), which is hereby incorporated by reference in its entirety). All candidate retinoid-response genes were used initially as probes against the original RNA samples used to construct the cDNA libraries. Independent samples of RNA were also obtained from RASMC treated for 3, 6, 12, 24, 48, 72, 96, and 120 hr with 2×10⁻⁶ mol/L atRA to obtain a temporal pattern of expression for each positive clone. In some experiments the effects of cycloheximide (CHX, 2.5 μg/ml) on atRA-induced gene expression were examined. This concentration of CHX reduces de novo protein synthesis by more than 90% without any signs of overt cytotoxicity. Total RNA was size-fractionated in 1.2% agarose-formaldehyde gels, transferred to nylon membranes and then hybridized and washed following standard procedures (ExpressHyb™, CLONTECH, Palo Alto, Calif.). A rat glyceraldehyde phosphate dehydrogenase (GAPDH) or 18 S probe was used as an internal control for RNA loading. Autoradiography was performed and exposure times varied so that band intensities could be reliably quantitated using NIH Image 1.60 analysis software.

In Situ Hybridization

Cross-sections (5 μm) from non-injured rat common carotid arteries were used to determine the spatial mRNA expression of two cloned retinoid-response genes. Rat tissue transglutaminase (290 nt fragment) and rat α₈ integrin (246 nt fragment) were linearized and used in in vitro transcription assays to generate ³³P-labeled sense and antisense riboprobes (MaxiScript, Ambion, Austin, Tex.). A 275 nt sense or antisense fragment of the rat SM22 cDNA was used as a positive control for SMC expression. Paraffin-embedded, paraformaldehyde-fixed tissue sections were deparaffinized, rehydrated, and treated with proteinase K at 37° C. for 6 minutes. Hybridization with ˜3×10⁷ cpm of probe per ml of hybridization solution was performed overnight at 52° C. in a humidified chamber. Slides were then washed to remove unbound probe, treated with 10 μg/ml RNase A at 37° C. for 30 minutes, dehydrated, air-dried, and dipped in emulsion (Kodak NTB2, Rochester, N.Y.). After 1 week, slides were developed in Kodak D19 developer, fixed, and counter-stained with Mayer's hematoxylin. Darkfield images were captured with a Polaroid digital camera using Adobe Photoshop.

All-Trans Retinoic Acid Induces a Novel Retinoid-Response Gene Set in Vascular SMC

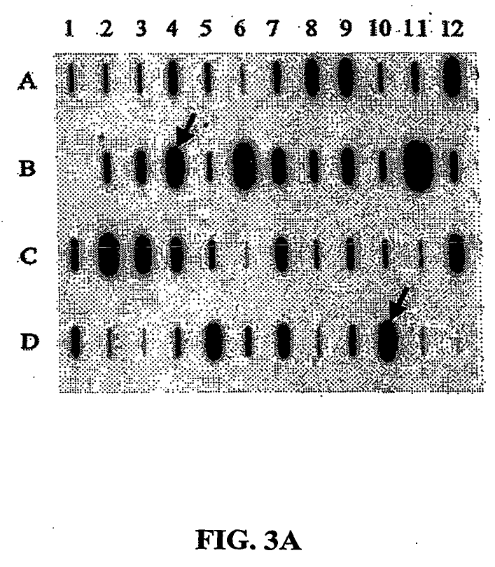

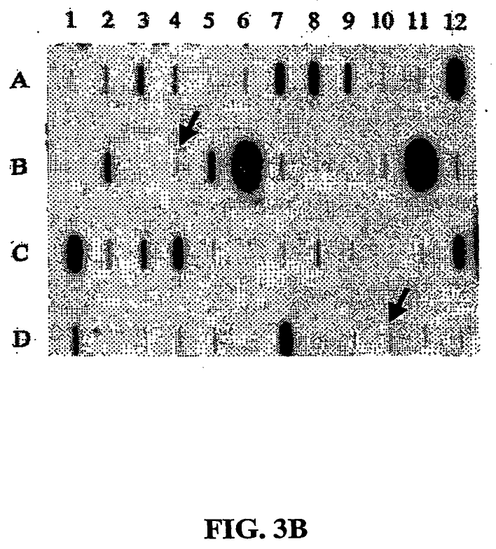

Of the 300 clones picked for differential screening, 47 (15%) appeared to be up-regulated with all-trans retinoic acid (“atRA”) treatment. An example of a differential screen is shown in FIGS. 3A-B. Each of the 47 putative retinoid-response genes was then subjected to a “mini” Northern blot containing the original RNA samples used for cDNA library construction. This analysis verified the atRA-inducibility of 14 (30%) clones. 13-cis RA and the synthetic retinoid 4-Hydroxyphenylretinamide also elevated the expression of this gene set. The remaining 33 clones showed either less than 2-fold differences in expression or no detectable expression. Analysis of a “reverse library screen” for atRA-mediated gene repression failed to reveal any clones exhibiting a consistent down-regulated pattern of expression. An annotated listing of the 14 atRA-inducible clones is provided in Table 2 below.

TABLE 2
SMC Retinoid-Responsive Genes¹

| | | | Peak Fold | | |
|---|---|---|---|---|---|
| Gene² | Accession Number³ | mRNA size | Induction⁴ | CHX Inhibit | Putative Function |
| SSAT | L10244 | 1.8 kb | 4.8 (48 hr) | Yes | Growth Suppression |
| SSeCKS | U23146 | 6.0 kb | 4.8 (12 hr) | No | Growth Suppression |
| Epithelin | X62322 | 3.4 kb | 2.9 (48 hr) | Yes | Growth Suppression |
| α₈-integrin | AF148797 | 6.0 kb | 2.4 (72 hr) | Yes | Differentiation |
| VCAM-1 | M84488 | 3.8 kb | 2.5 (96 hr) | Yes | Differentiation |
| tTG | AF106325 | 3.6 kb | >4.0 (12 hr) | No | Differentiation/Apoptosis |
| LDH-B | U07181 | 1.9 kb | 4.6 (24 hr) | Yes | Apoptosis |
| LOX-1 | AB005900 | 4.0 kb | 7.2 (3 hr) | No | Apoptosis |
| RDH | AF061743 | 3.5 kb | >2.8 (48 hr) | Yes | Retinoid Metabolism |
| Cathepsin-L | S85184 | 2.2 kb | 3.5 (72 hr) | Yes | Retinoid Metabolism |
| Ceruloplasmin | L33869 | 4.8 kb | 2.0 (72 hr) | Yes | Anti-oxidant |
| Importin α | AJ130946 | 3.0 kb | 4.0 (12 hr) | Yes | Nuclear Chaperone |
| Endolyn | AJ238574 | 4.0 kb | 2.0 (96 hr) | No | Lysosome Biogenesis |
| SALF | AF026169 | 6.5 kb | 4.1 (3 hr) | No | Membrane Trafficking |

¹Italicized genes represent those previously shown to be retinoid-inducible
²Abbreviated gene names are spermidine/spermine N¹-acetyltransferase (SSAT); src suppressed C kinase substrate (SSeCKS); vascular cell adhesion molecule (VCAM-1); tissue transglutaminase (tTG); lactate dehydrogenase (LDH-B); lectin-like oxidized LDL receptor (LOX-1); retinol dehydrogenase (RDH); Stoned B/TFIIAα/β-like Factor (SALF).
³Each of the Genbank Accessions is incorporated herein in its entirety.
⁴Densitometric measurements taken at peak time of expression (in parenthesis) following atRA stimulation (refer to FIG. 3A-B). Peak fold increases are relative to the 0 time point except for tTG and RDH where the 3 hr time points were used as “baseline”

Cycloheximide and Temporal Expression Studies Reveal Two Classes of Retinoid-Response Gene

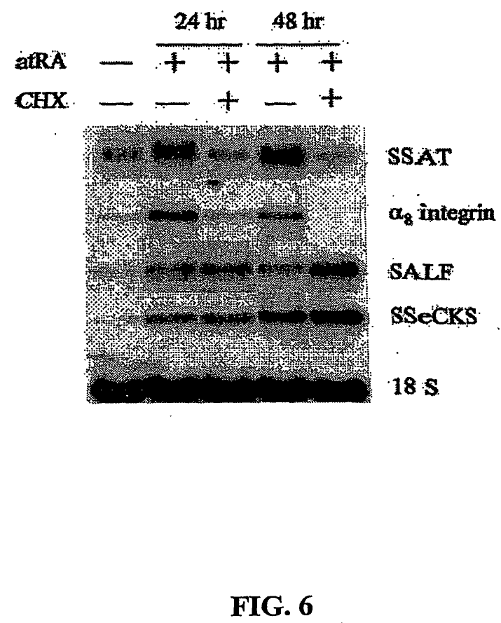

To determine whether atRA-induced gene expression required de novo protein synthesis, rat aorta smooth muscle cell tissue was incubated with atRA in the absence or presence of cycloheximide (“CHX”) and then measured the expression of each atRA-inducible gene. FIG. 6 shows that atRA-induced spermidine/spermine N¹-acetyltransferase (“S SAT”) and α8-integrin mRNA were attenuated with CHX. On the other hand, atRA-induced Stoned B/TFIIAα/β-like factor (“SALF”) and Src Suppressed C Kinase Substrate (“SSeCKS”) mRNA were not blocked with CHX. In fact, there was a slight super-induction of these genes with CHX, shown in FIG. 6, which is a common characteristic of immediate early genes. Table 2, above, summarizes the sensitivity of each atRA-inducible gene to CHX.

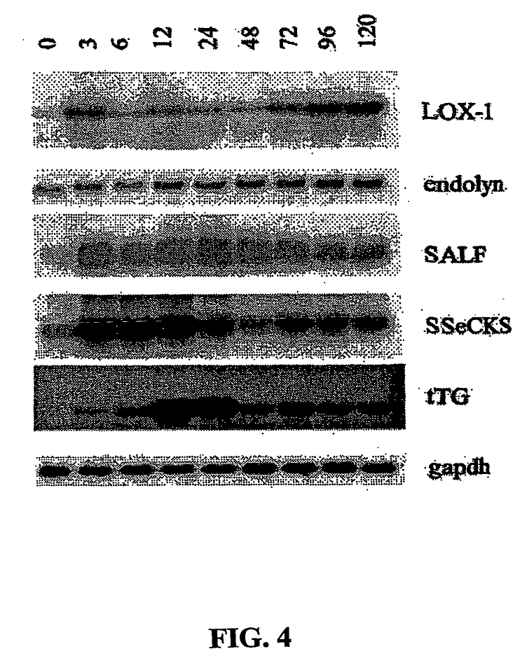

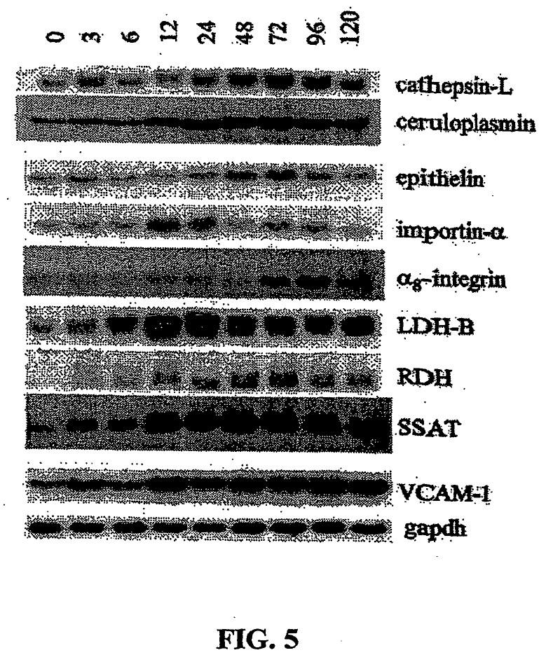

By definition, genes whose induction does not require de novo protein synthesis are said to be immediate early genes and, in general, the onset of induction of such genes is rapid (within a few hours) Sheng et al., “The Regulation and Function of C-Fos and Other Immediate Early Genes in the Nervous System,” Neuron 4:477-485 (1990), which is hereby incorporated by reference in its entirety). In contrast, genes whose elevated expression requires de novo protein synthesis display somewhat slower kinetics of expression and are thus referred to as delayed response genes. The temporal pattern of each gene's mRNA expression was examined over a 5-day course of atRA stimulation. FIG. 4 shows the expression kinetics of LOX-1, endolyn, SALF, SSeCKS, and tissue transglutaminase (tTG) whose atRA-inducibility occurred in the presence of CHX (FIG. 6 and Table 2). In general, the onset of mRNA induction with these genes occurred within a few hours of atRA administration. In contrast, atRA-stimulated cathepsin-L, ceruloplasmin, epithelin, importin α, α₈-integrin, LDH-B, RDH, SSAT, and VCAM-1 mRNA, whose induction was blocked with CHX (FIG. 6 and Table 2), showed somewhat slower kinetics of peak expression, shown in FIG. 5 and Table 2. Taken together, these results allowed the categorization of each clone as either an immediate early retinoid-response gene (FIG. 4) or a delayed retinoid-response gene (FIG. 5).

Two Retinoid-Response Genes Display a Vascular SMC-Restricted Pattern of Expression

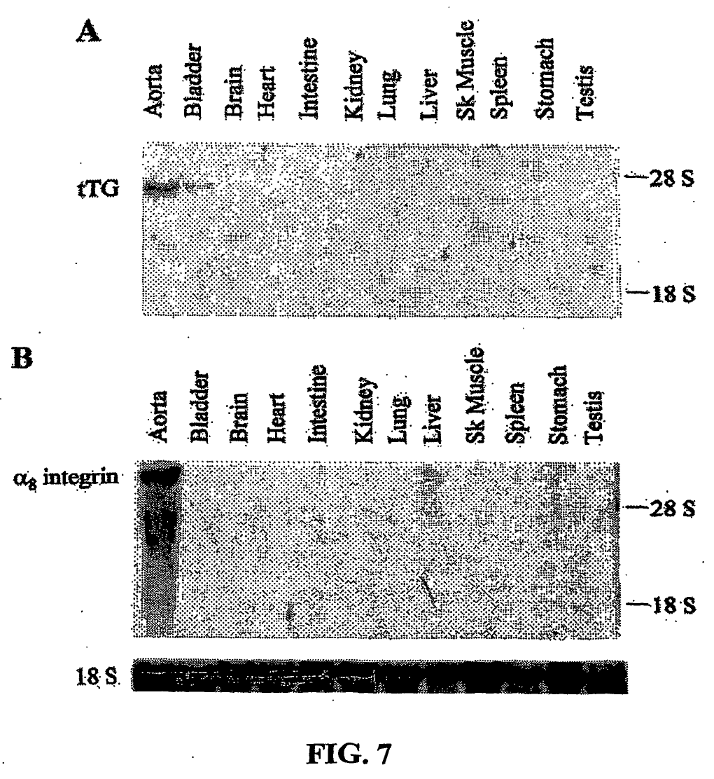

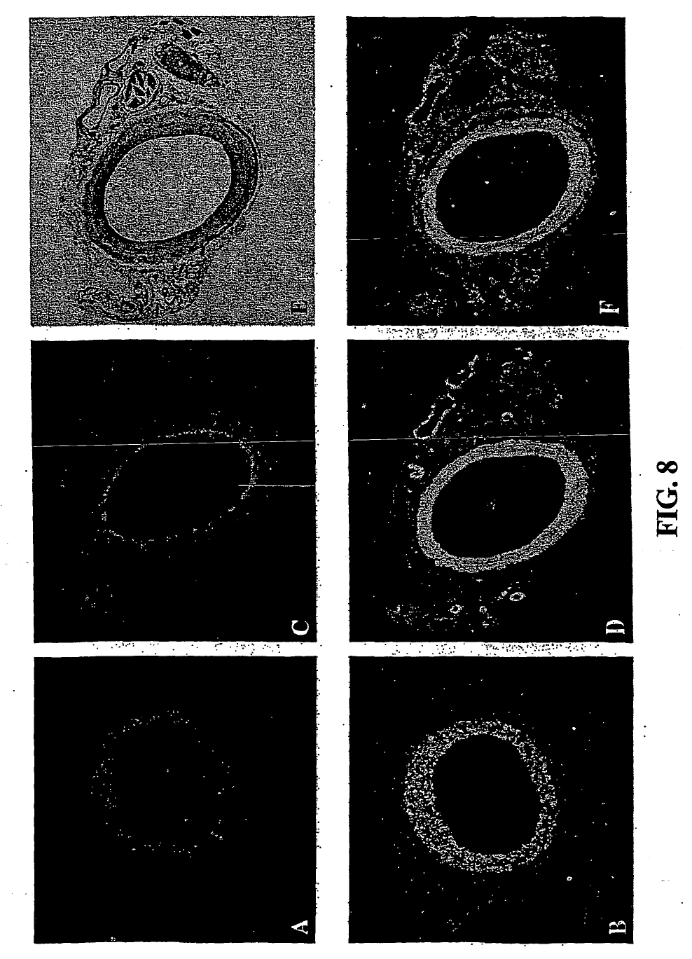

To determine the tissue distribution of retinoid-response genes, the mRNA expression of each gene was examined in adult rat tissues. Data presented in FIGS. 7A-B show the tissue distribution of two retinoid-response genes, tTG and α₈-integrin. The expression of tTG was highest in adult aorta with barely detectable levels in the bladder, shown in FIG. 7A. The expression of α₈-integrin mRNA was also highly restricted to aortic tissue with virtually no discernible transcripts observed in other tissues, shown in FIG. 7B. The localized expression of tTG and α8-integrin mRNA in normal and balloon-injured rat carotid artery was also examined. Normal (non-ballooned) carotid arteries showed prominent hybridization signals to tTG, α₈-integrin, and the SMC-restricted gene, SM22. All three of these transcripts could also be demonstrated in both the medial and neointimal layers of balloon-injured rat carotid arteries, shown in FIGS. 8A-F. In contrast, there was only slight hybridization to adventitial cells, shown in FIG. 8. These results demonstrate vascular SMC-restricted expression of two retinoid response genes whose level of mRNA decreases in culture, but could be prominently induced with retinoid treatment.

Discussion

Retinoids are thought to exert their pleiotropic effects via binding and activating retinoid receptors, which modulate gene expression and hence alter a cell's phenotype. Previously, it was shown that cultured SMC express 5 of the 6 retinoid receptors and exhibit retinoid receptor activity in vitro Miano et al., “Retinoid Receptor Expression and All-Trans Retinoic Acid-Mediated Growth Inhibition in Vascular Smooth Muscle Cells,” Circulation 93:1886-1895 (1996), which is hereby incorporated by reference in its entirety). These results and the known inhibitory effects of retinoids on SMC growth and neointimal formation (Neuville et al., “Retinoids and Arterial Smooth Muscle Cells,” Arterioscler. Thromb. Vasc. Biol. 20:1882-1888 (2000); Miano et al., “Retinoids: Versatile Biological Response Modifiers of Vascular Smooth Muscle Phenotype,” Circ. Res. 87:355-362 (2000), which are hereby incorporated by reference in their entirety) prompted the present study. The results of the subtractive screening disclosed 14 retinoid-inducible genes, which were categorized as either immediate early or delayed based on sensitivity to CHX and expression kinetics, as shown in FIGS. 4, 5, and 6, and Table 2, above. It is likely these results were achieved, in part, because of the design of the subtractive hybridization screen, namely the inclusion of two independent time points during initial cDNA library construction. Because gene expression may vary temporally, it is possible that combining cDNAs from two time points could increase the number of retinoid-responsive genes in the tester cDNA pool. The fact that this gene set could be categorized into early and delayed-responsive classes demonstrates the utility of this modification.

The rapid accumulation of immediate early retinoid-response genes suggests that induction likely occurs through the direct binding of ligand-activated retinoid receptors to retinoid receptor response elements located in a gene's regulatory region (Leid et al., “Multiplicity Generates Diversity in the Retinoic Acid Signalling Pathways,” Trends Biochem. Sci. 17:427433 (1992), which is hereby incorporated by reference in its entirety). This is certainly the case with the immediate early retinoid-response gene tTG, which contains a functional retinoic acid response element located 1.7 kilobases upstream of the start site of transcription Nagy et al., “Identification and Characterization of a Versatile Retinoid Response Element (Retinoic Acid Receptor Response Element-Retinoid X Receptor Response Element) in the Mouse Tissue Transglutaminase Gene Promotor,” J. Biol. Chem. 271:4355-4365 (1996), which is hereby incorporated by reference in its entirety). Future studies should determine whether the other immediate early retinoid response genes cloned in this report are similarly regulated by ligand-activated retinoid receptors.

The majority of genes cloned in this report appear to require protein synthesis for atRA-inducibility. These so-called delayed retinoid-response genes, shown in FIG. 5, are likely induced in an indirect manner through an intermediary factor(s) whose expression and/or activity requires de novo protein synthesis. It is possible that some of the intermediary factors may themselves be direct targets of RA. For example, several homeobox genes, which encode for transcription factors involved in cell differentiation and growth, are known targets of activated retinoid receptors (Langston et al., “Retinoic Acid-Responsive Enhancers Located 3′ of the Hox A and Hox B Homeobox Gene Clusters: Functional Analysis,” J. Biol. Chem. 272:2167-2175 (1997), which is hereby incorporated by reference in its entirety). The possibility should also be considered that elevated delayed retinoid-response gene mRNA expression may be a consequence of some post-transcriptional event (e.g., mRNA stability). Alternatively, delayed retinoid-response genes may require the activity of a constitutively expressed labile protein.

Most of the genes in the present study have heretofore been unrecognized as retinoid-inducible (see Table 2, above, for identification of previously known retinoid inducible genes). This indicates that SMC respond in a unique manner to atRA stimulation and suggests that the genes induced by atRA represent important mediators of SMC phenotype. Indeed, as shown in Table 2, it was possible to classify this gene set into functional groups associated with known retinoid-mediated activities. For example, atRA has been shown to support a differentiated phenotype in vascular SMC (Neuville et al., “Retinoids and Arterial Smooth Muscle Cells,” Arterioscler. Thromb. Vasc. Biol. 20:1882-1888 (2000); Miano et al., “Retinoids: Versatile Biological Response Modifiers of Vascular Smooth Muscle Phenotype,” Circ. Res. 87:355-362 (2000), which are hereby incorporated by reference in their entirety). Among the genes cloned in this report, three could be categorized as differentiation-related (VCAM-1, tTG, and α₈-integrin). VCAM has been shown to promote the expression of SMC differentiation markers in cultured human SMC (Duplaa et al., “The Integrin Very Late Antigen-4 is Expressed in Human Smooth Muscle Cells: Involvement of α₄ and Vascular Cell Adhesion Molecule-1 During Smooth Muscle Cell Differentiation,” Circ. Res. 80:159-169 (1997), which is hereby incorporated by reference in its entirety). Both tTG and α₈-integrin are highly expressed in SMC of blood vessels Thomázy et al., “Differential Expression of Tissue Transglutaminase in Human Cells: An Immunohistochemical Study,” Cell Tissue Res. 255:215-224 (1989); Schnapp et al., “Sequence and Tissue Distribution of the Human Integrin α₈ Subunit: a β₁-Associated α Subunit Expressed in Smooth Muscle Cells,” J. Cell Sci. 108:537-544 (1995), which are hereby incorporated by reference in their entirety), but are down-regulated when SMC are cultured in vitro (Lee et al., “Modulation of Large Conductance Ca²⁺-Activated K⁺ Channel by Gah (Transglutaminase II) in the Vascular Smooth Muscle Cells,” Pflugers Arch. 433:671-673 (1997), which is hereby incorporated by reference in its entirety). tTG is thought to promote structural integrity to blood vessels through its cross-linking activity (Thomázy et al., “Differential Expression of Tissue Transglutaminase in Human Cells: An Immunohistochemical Study,” Cell Tissue Res. 255:215-224 (1989), which is hereby incorporated by reference in its entirety). α₈-integrin dimerizes with the β₁-integrin subunit, itself an atRA-induced gene Medhora, “Retinoic Acid Upregulates β₁-Integrin in Vascular Smooth Muscle Cells and Alters Adhesion to Fibronectin,” Am. J. Physiol. 279:H382-H387 (2000), which is hereby incorporated by reference in its entirety), and together the dimer serves as a receptor for fibronectin, vitronectin and tenascin (C Schnapp et al., “The Human Integrin α8β₁ functions as a Receptor for Tenascin, Fibronectin, and Vitronectin,” J. Biol. Chem. 270:23196-23202 (1995), which is hereby incorporated by reference in its entirety). Interestingly, α₈-integrin mRNA is expressed abundantly in aortic SMC, but appears to be virtually absent in other SMC-containing tissues, see FIG. 7B. This finding is similar to that of the aortic preferentially expressed gene (Hsieh et al., “APEG-1, a Novel Gene Preferentially Expressed in Aortic Smooth Muscle Cells, is Down-Regulated by Vascular Injury,” J. Biol. Chem. 271:17354-17359 (1996), which is hereby incorporated by reference in its entirety) and suggests a potentially unique mode of transcriptional regulation for these vascular SMC-restricted genes.

In addition to genes associated with the SMC differentiation program, several genes were cloned whose encoded proteins influence cell growth and death (see Table 2). The growth-related genes included SSAT, an enzyme involved in the synthesis of putrescine (a substrate for tTG) (Ichimura et al., “Significant Increases in the Steady States of Putrescine and Spermidine/Spermine N¹-Acetyltransferase mRNA in HeLa Cells Accompanied by Growth Arrest,” Biochem. Biophys. Res. Comm. 243:518-521 (1998), which is hereby incorporated by reference in its entirety), epithelin, an understudied anti-mitogen (Plowman et al., “The Epithelin Precursor Encodes Two Proteins With Opposing Activities on Epithelial Cell Growth,” J. Biol. Chem. 267:13073-13078 (1992), which is hereby incorporated by reference in its entirety), and SSECKS (see Table 2). SSeCKS was first discovered in a screen for tumor suppressor genes in NIH 3T3 cells where its levels were found to decrease more than 15-fold in src-, ras-, andfos-transformed cells (Lin et al., “Isolation and Characterization of a Novel Mitogenic Regulatory Gene, 322, Which is Transcriptionally Suppressed in Cells Transformed by Src and Ras,” Mol. Cell. Biol. 15:2754-2762 (1995), which is hereby incorporated by reference in its entirety). The function of SSeCKS is slowly emerging as a large membrane docking protein with binding sites for numerous signaling proteins including protein kinases A and C Lin et al., “A Novel Src- and Ras-Suppressed Protein Kinase C Substrate Associated with Cytoskeletal Architecture,” J. Biol. Chem. 271:28430-28438 (1996), which is hereby incorporated by reference in its entirety) and, more recently, cyclin D1 whose expression is decreased when SSeCKS is constitutively active (Lin et al., “SSeCKS, a Major Protein Kinase C Substrate with Tumor Suppressor Activity, Regulates G1-S Progression by Controlling the Expression and Cellular Compartnentalizafion of Cyclin D,” Mol. Cell. Biol. 20:7259-7272 (2000), which is hereby incorporated by reference in its entirety). Three of the atRA-induced genes encode for proteins that play a role in the apoptotic program. tTG and LDH-B have been shown to mediate apoptosis in a variety of cellular contexts (Miyashita et al., “Cytotoxicity of Some Oxysterols on Human Vascular Smooth Muscle Cells Was Mediated by Apoptosis,” J. Atheroscler. Thromb. 4:73-78 (1997); Ou et al., “Retinoic Acid-Induced Tissue Transglutaminase and Apoptosis in Vascular Smooth Muscle Cells,” Circ. Res. 87:881-887 (2000), which are hereby incorporated by reference in their entirety). The role of the oxidized LDL receptor, LOX-1, in this process is less intuitive. However, a recent report provided evidence for LOX-1 in mediating the phagocytosis of apoptotic endothelial cells (Oka et al., “Lectin-Like Oxidized Low-Density Lipoprotein Receptor 1 Mediates Phagocytosis of Aged/Apoptotic Cells in Endothelial Cells,” Proc. Natl. Acad. Sci. USA 95:9535-9540 (1998), which is hereby incorporated by reference in its entirety). Thus, in addition to serving a role in oxidized LDL signaling, LOX-1 may have a secondary function in clearing apoptotic cells in an environment where programmed cell death has been initiated.

The list of atRA-inducible genes in Table 2 also includes a protease (cathepsin-L) involved with RXRA cleavage; a retinol dehydrogenase gene that might serve to buffer intracellular levels of retinal; an anti-oxidant (ceruloplasmin) that may buffer intracellular free radicals, especially superoxide anion (Goldstein et al., “Ceruloplasmin: A Scavenger of Superoxide Anion Radicals,” J. Biol. Chem. 254:4040-4045 (1979), which is hereby incorporated by reference in its entirety); a lysosomal membrane-associated protein (endolyn) that is thought to participate in lysosomal biogenesis (Inrke et al., “Endolyn is a Mucin-Like Type I Membrane Protein Targeted to Lysosomes by its Cytoplasmic Tail,” Biochem. J. 345:287-296 (2000), which is hereby incorporated by reference in its entirety); a newly discovered gene (SALF) that is proposed to play a role in membrane trafficking (Upadhyaya et al., “Identification of a General Transcription Factor TFIIAα/β Homolog Selectively Expressed in Testis,” J. Biol. Chem. 274:18040-18048 (1999), which is hereby incorporated by reference in its entirety); and a gene encoding for a nuclear chaperone protein (importin a), which could conceivably assist activated retinoid receptors in the generation of a new transcriptome by transporting critical nuclear proteins necessary for gene transcription (Görlich, “Transport Into and Out of the Cell Nucleus,” EMBO.J. 17:2721-2727 (1998), which is hereby incorporated by reference in its entirety).

### Example 2

### Sequencing and Characterization of a RISC Gene

During the cloning and expression studies of several retinoid response genes whose gene sequences matched known cDNAs, a cDNA clone was uncovered that was not found in any of the genomic databases. This gene was further characterized as follows.

cDNA Cloning, Sequencing, and Bioinformatic Analysis

A RASMC cDNA library was screened with a 405 nt fragment of RISC to isolate longer fragments of the RISC cDNA. 5′ rapid amplification of cDNA ends (“RACE”) was performed using a 5′ RACE kit (Gibco/BRL Inc., Grand Island, N.Y.) with gene-specific primers. 3′ untranslated (UTR) sequences were obtained by reverse transcriptase polymerase chain reaction (RT-PCR) (First Strand Synthesis Kit, Amersham Pharmacia Biotech, Piscataway, N.J.) of atRA-stimulated RASMC using oligo dT and RISC-specific primers. Phage clones and RACE products were sequenced on both strands (University of Rochester Core Nucleic Acid Laboratory) and analyzed with the GCG suite of software programs (GCG, Version 10.1, Madison Wis.) and BLASTN at the National Center for Biotechnology Information website. RISC peptide sequence comparison was performed using BlastP. Molecular weight and pI were determined using ProtParam. The PROSITE pattern, PROSITE profile, BLOCKS, ProDom, PRINTS, and Pfam databases were scanned using MOTIF. Signal peptide prediction was performed using SignalP V2.0. Multiple sequence alignments were performed using the pileup command in GCG. Serine carboxypeptidase active site consensus motifs were obtained from PROSITE. Finally, the Online Mendelian Inheritance in Man Morbid Map was consulted to determine whether human RISC was associated with a known genetic disease.

Analysis of the rat RISC cDNA using BLAST revealed numerous mouse expressed sequence tags (ESTs) with similarity to rat RISC. ESTs were aligned using the pileup command in GCG's Wisconsin package, with the rat cDNA as the template. A consensus sequence was determined using the pretty command with the pileup file as the input. The consensus sequence was determined from at least five overlapping ESTs when possible. A region between 600 and 900 nt was underrepresented (<4 ESTs) in the EST pool and thus no perfect consensus was reached. Here ambiguous nucleotides were edited using rat RISC as a guide to preserve the reading frame. The ESTs used to assemble the mouse RISC cDNA are AA612086, AA726687, AI118955, AI854112, AI875629, AI957256, AW107478, AW744471, BE850553, BE852371, BF016593, BF182152, C89145, WI 0703, each of which is hereby incorporated by reference in its entirety.

In Vitro Transcription/Translation

RISC protein was in vitro translated with the TNT T7 Quick Coupled Transcription/Translation System (Promega Inc, Madison, Wis.). A fragment corresponding to the full-length open reading frame of RISC was generated by RT-PCR of total RNA isolated from RASMC treated with atRA for 96 hr using restriction enzyme-clamped (underlined sequence) RISC-specific primers: forward primer, 5′-GATACGTCGACCTGAGGCGGGGTTTTCATC-3′ (SEQ ID No: 66) and reverse primer, 3′-GATACGATATCTGTGATGGAGCCGAGGATGC-3′ (SEQ ID No: 67). Template cDNA was obtained by subcloning the PCR-amplified product into pBluescriptII SK+ (Stratagene, La Jolla, Calif.). A Luciferase T7 cDNA was used as a positive control. In vitro transcription-translation was carried out with 1 μg of plasmid DNA in 50 μl of reaction mixture supplemented with 50 μCi of [³⁵S] methionine (Amersham Pharmacia, Piscataway, N.J.) and various amounts of TNT T7 Quick Master Mix (Promega Inc, Madison, Wis.) for 90 min at 30° C. 10 μl of the products were separated by 10% SDS-polyacrylamide gel electrophoresis, and dried gels were analyzed by autoradiography.

Confocal Microscopy of RISC-His Tagged Fusion Protein

RT-PCR was performed using the ProSTAR Ultra HF RT-PCR System (Stratagene, La Jolla, Calif.) employing a pair of RISC-specific primers: same forward primer as above; reverse primer, 5′-GATACTCTAGACTCCTGCTGAGTAACCAG-3′ (SEQ ID No: 68). The amplification product, corresponding to the open reading frame of RISC less the stop codon at nt 1376-1378, was subcloned into pEF1/V5-His (Invitrogen, Carlsbad, Calif.) to generate RISC-His. Sub-cellular localization of RISC-His fusion protein was appraised by confocal immunofluorescence microscopy. Briefly, COS-7 cells were grown to 60-80% confluence on 4-well chamber slides and transfected with RISC-His using LipofectAMINE PLUS reagents. The vector without insert was also transfected in parallel as a mock control. The cells were then allowed to grow for 24 or 48 h in complete medium. After washing with cold-PBS, the cells were fixed in cold methanol/acetone (1:1) mixture at −20° C. for 10 min. The slides were then incubated at room temperature for 1 hr in the presence of anti-His monoclonal antibody (Invitrogen, Carlsbad, Calif.) diluted 1:200 in PBS, washed with PBS, and finally incubated for 1 hr in the presence of FITC-conjugated secondary antibody (Pierce, Rockford, Ill.) diluted 1:100 in PBS. Cells were then stained with the DNA fluorochrome, TO-PRO-3 iodide (Molecular Probes, Eugene, Oreg.) to view the cell nucleus. Slides were coverslipped under aqueous mounting medium and viewed with an Olympus Fluoview FV 300 confocal microscope (Melville, N.Y.). Voltage settings were kept constant between RISC-His and mock-transfected cells during imaging. For each experiment, primary and secondary antibody controls were also included. Final images were captured and processed with Adobe Software.

Western Blotting

The secretory property of RISC was examined by Western blotting. COS-7 cells were grown in 6-well plates to 60-80% confluence and then transfected with RISC-His using LipofectAMINE PLUS reagents (Gibco BRL/Life Science Technologies, Rockville, Md.). 24 hr after transfection, the cells were re-fed with 0.1% FBS or serum free medium and incubated for another 24 or 48 hr. The conditioned medium and extracts prepared from transfected cells were analyzed for expressed RISC-His protein by Western blotting using an anti-His monoclonal antibody (Invitrogen, Carlsbad, Calif.). The proteins from the medium (non-concentrated) and cell lysates were separated on a 10% SDS-polyacrylamide gel, transferred onto nitrocellulose membrane and immunoblotted with anti-His monoclonal antibody diluted 1:2000. Immunocomplexes were detected with a secondary antibody conjugated to horseradish peroxidase (Pierce, Rockford, Ill.) and visualized with SuperSignal West Pico Luminol/Enhancer Solution (Pierce, Rockford, Ill.).

Northern Blotting Studies

10-20 μg of total RNA from RASMC, PAC-1 SMC, hCASMC or rat tissues was fractionated on 1.1% agarose gel in the presence of 0.66 mol/L formaldehyde, transferred to nylon membrane, and hybridized with a 405 nt RISC cDNA probe labeled with α-³²P-dCTP. For human tissue Northern blotting, a Human Multiple Tissue Northern Blot was obtained from CLONTECH and hybridized with a human RISC DNA probe, cloned from a human BAC (AC007114; obtained from Research Genetics, Inc., Huntsville, Ala.) by PCR. Hybridizations were carried out in Rapid-hyb buffer (Amersham Life Science, Piscataway, N.J.) or Expresshyb Hybridization Solution (CLONTECH, Palo Alto, Calif.) containing labeled probe (˜2×10⁶ cpm/ml) at 62-68° C. for 1-2 h or overnight, depending on the probe used. The blots were then washed under stringent conditions and exposed to Kodak XAR films for different lengths of time. Housekeeping genes used as internal controls for equal RNA loading included glyceraldehyde 3-phosphate dehydrogenase (GAPDH), 18 S rRNA, and β-actin.

In Situ Hybridization Studies

Selective rat tissues were used to determine RISC mRNA expression in vivo. The original 405 nt rat RISC cDNA fragment obtained from the SSH screen was cloned into pBluescript SK and then linearized with either Bam HI (for antisense riboprobe) or XhoI (for sense riboprobe). [³³P]UTP-labeled riboprobes were synthesized by in vitro transcription using MAXIscript in vitro transcription Kit (Ambion, Austin, Tex.). Both antisense and sense riboprobes were assayed and the latter used as a control for specificity of the signal. Paraffin-embedded, formaldehyde-fixed tissue sections were deparaffinized and treated with 5 μg/ml proteinase K at 37° C. for 6 min. Hybridization with 3×10⁷ cpm of probe per milliliter of hybridization solution was performed overnight at 52° C. in a humidified chamber. Slides were washed to remove unbound probe, treated with RNase A, dehydrated with ethanol, air-dried, and dipped in emulsion (Kodak NTB2). After one week, slides were developed in Kodak D19 developer. Dark-field and bright-field images were taken with an Olympus digital camera and processed in Adobe Photoshop. The final composite of images was assembled in FreeHand (Macromedia, San Francisco, Calif.).

Chromosomal Mapping of Rat RISC

The location of RISC in the rat genome was determined with a rat/hamster radiation hybrid (RH) panel (Research Genetics, Huntsville, Ala.) using rat-specific primers. The sequence of the forward primer was 5′-CTCTTCTTCCCGACTCTACCAT-3′(SEQ ID No: 69); the sequence of the reverse primer was 5′-GAACTTGTGATGGAGCCGAGG-3′ (SEQ ID No: 70). All RH clones except numbers 1, 20, 35, 38, 60, 90, 101-106 were used. PCR products were separated on a 1.2% agarose gel and a vector was obtained by scoring resolved PCR products as negative=0, positive=1, or ambiguous=2. The resulting vector was submitted to the Rat RH Map Server at the Rat Genome database to obtain the chromosomal location of RISC.

Results: Differential Cloning of a Novel Retinoid Response Gene in Vascular SMC

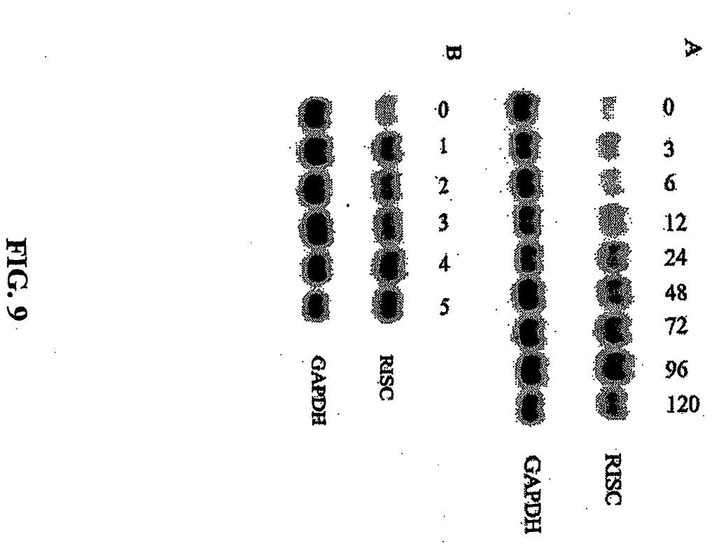

During the characterization of this gene set, described in Example 1, a cDNA clone was found that was not identified in any of the genomic databases but showed significant homology to several serine carboxypeptidase ESTs. This clone was designated as “RISC”, described supra. Low stringency Southern blotting of rat genomic DNA suggests that RISC is a single copy gene. FIG. 9A shows the induction of a ˜2.1 kb RISC transcript beginning 3 hr after atRA administration. RISC mRNA levels increased progressively over a 5 day time course in which fresh atRA was applied daily, as shown in FIG. 9A. A similar course of RISC mRNA induction was observed in cells treated with only one application of atRA, shown in FIG. 9B. Also observed were increases in RISC mRNA following atRA stimulation of PAC-1 SMC and hCASMC.

To obtain additional sequence data, a rat aortic SMC cDNA library was screened with the 405 nt SSH-derived fragment and sequenced 12 independent clones. Assembly of these clones, coupled with 5′ RACE and 3′ RT-PCR, led to the deduced RISC sequences shown in FIG. 2. FIG. 1 shows the amino acid sequence and spacing homology of the substrate recognition domain and catalytic triad of mammalian RISC (domains 1-IV) to several evolutionarily distant serine carboxypeptidases.

Several mouse RISC ESTs have been assembled into a cDNA sequence (GenBank Accession Number AF330052) which is 93% homologous to the open reading frame of rat RISC (92% amino acid sequence identity/similarity). The human ortholog of rat RISC (named Human Serine Carboxypeptidase Precursor 1) is disclosed at GenBank Accession number AF282618, which is hereby incorporated by reference in its entirety). The rat RISC cDNA is 80.9% homologous to the open reading frame of human RISC (with 82% amino acid identity).

In Vitro Translation and Intracellular Localization of RISC

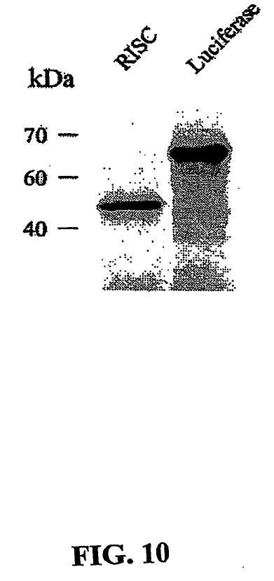

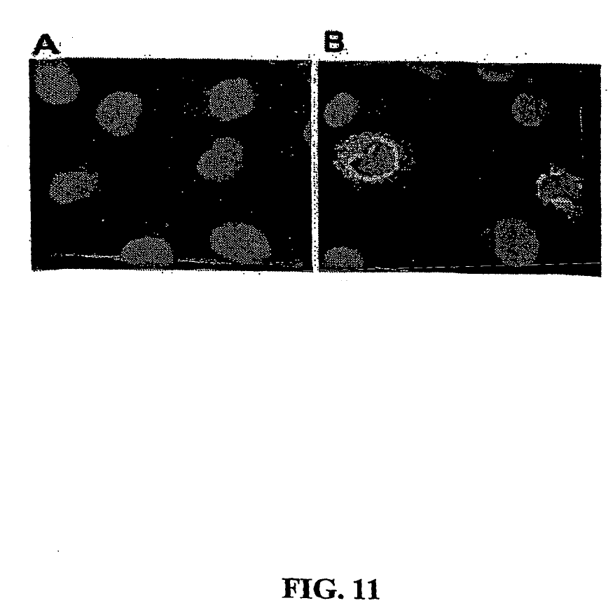

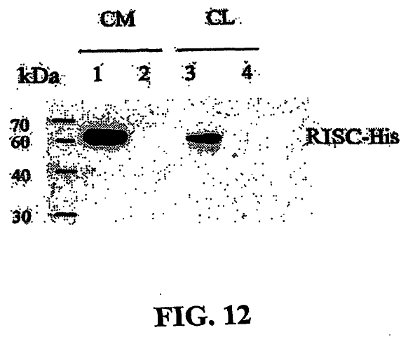

In vitro transcription-translation of rat RISC resulted in a protein with a molecular weight close to the predicted 51.2 kDa, as shown in FIG. 10. To determine the cellular localization of RISC, confocal immunofluorescence microscopy of COS-7 cells transfected with a RISC-His tag fusion protein was performed. These results, shown in FIGS. 11A-B, reveal a perinuclear distribution of the fusion protein that extends into the cytosol. Little or no nuclear accumulation of RISC was observed, as shown in FIG. 1B. Consistent with the presence of a putative N-terminal signal sequence, RISC-His is secreted from transfected COS-7 cells, shown in FIG. 12. Although the predicted RISC-His fusion protein is 55 kDa, the secreted and intracellular forms of the protein migrate at a molecular weight greater than 60 kDa, shown in FIG. 12. This could be due to post-translational modifications such as N-linked glycosylation (see FIG. 2).

RISC mRNA is Expressed in a Tissue-Restricted Manner

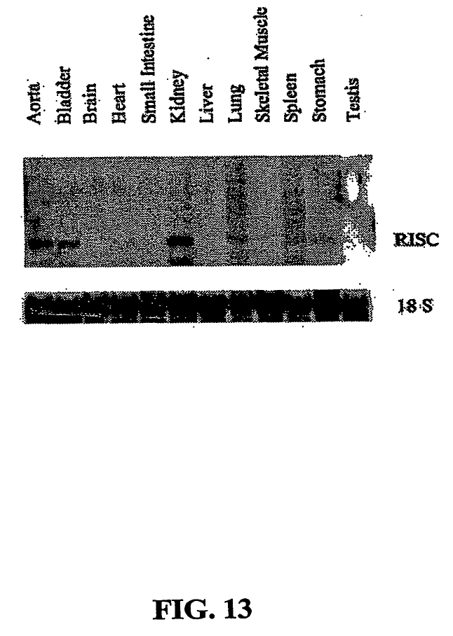

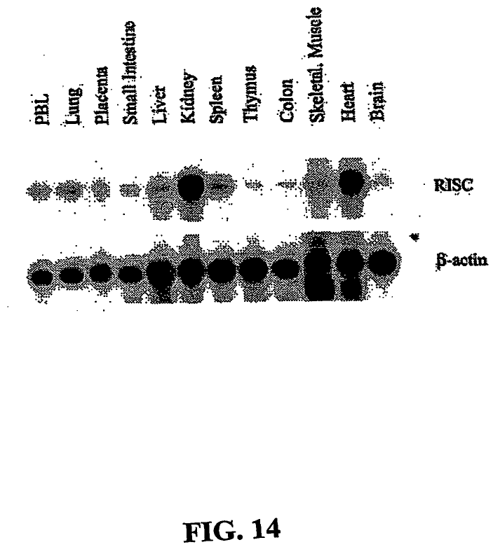

To determine the tissue distribution of RISC mRNA, Northern blots were performed on panels of rat and human tissues. As shown in FIG. 13, RISC mRNA is highly expressed in rat aorta, bladder, and kidney with lower levels in several other tissue types. A human polyA+ tissue blot, shown in FIG. 14, exhibits a similar restricted pattern of expression with highest levels in the kidney and heart (the latter is likely due to contaminating aortic tissue). Consistent with the kidney expression data, at least 10 ESTs with homology to RISC were obtained from various kidney cDNA libraries. These results indicate that high level RISC mRNA expression is tissue-restrictive suggesting that it may have functions specific for cells found in aorta, bladder, and kidney.

Spatial Localization of RISC mRNA in Rat Tissues

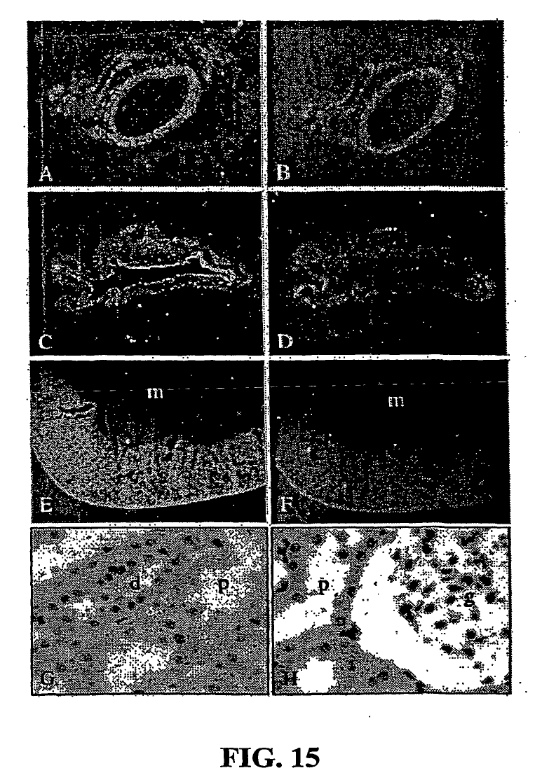

In situ hybridization studies, shown in FIGS. 15A-H, were carried out to determine the spatial distribution of RISC mRNA in various rat tissues. Rat RISC mRNA is modestly elevated in the rat aorta, shown in FIG. 15A, which may reflect the reduced cellularity of this tissue as compared to others and/or the heterogeneity that exists between SMC lineages of the aorta (Topouzis et al., “Smooth Muscle Lineage Diversity in the Chick Embryo: Two Types of Aortic Smooth Muscle Cell Differ in Growth and Receptor-Mediated Transcriptional Responses to Transforming Growth Factor-Beta,” Dev. Biol. 178:430445 (1996), which is hereby incorporated by reference in its entirety). FIG. 15C shows RISC expression was localized to the transitional epithelium of the bladder. In the kidney, RISC showed expression throughout the renal cortex with little or no hybridization signal in the renal medulla, shown in FIG. 15E. Careful examination of the cortical expression of RISC revealed that the transcript was confined largely to the epithelium of the proximal convoluted tubules, shown in FIGS. 15G-H. Glomerular cells, distal convoluted tubules, collecting ducts, juxtaglomerular cells, peri-tubular capillaries, and larger blood vessels showed only background hybridization signals, shown in FIGS. 15G-H. Consistent with the Northern blotting data, heart, liver, spleen, skeletal muscle, and brain showed only background RISC hybridization.

Rat RISC Maps to the Long Arm of Chromosome 10

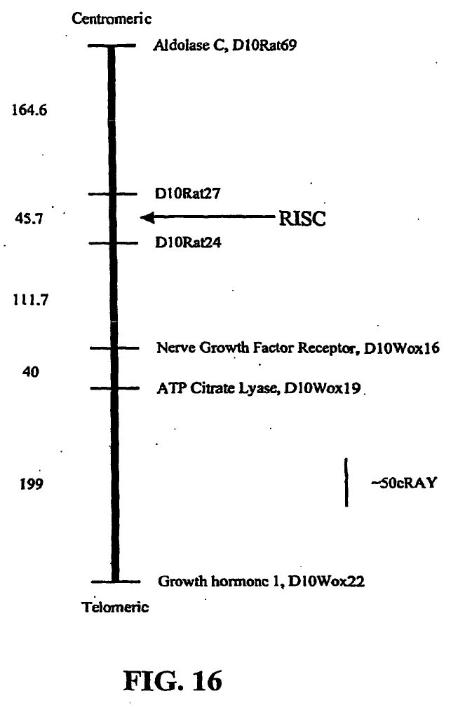

Using a rat-hamster RH panel, RISC was mapped to 10q31-10q32.1 of the rat genome, shown in FIG. 16. This region of chromosome 10q is syntenic to chromosome 17q23.1 in the human genome (see GenBank Accession Number NT_{—}010651, which is hereby incorporated by reference in its entirety). In silico analysis showed that the human ortholog of RISC is comprised of at least 13 exons spanning >25 kb of genomic sequence. Analysis of the Online Mendelian Inheritance in Man Morbid Map did not reveal a disease phenotype mapping to the region of human RISC.

Retinoids such as atRA have been shown to have desirable effects on SMC growth, migration, and differentiation both in vitro and in vivo (Miano et al., “Retinoids: Versatile Biological Response Modifiers of Vascular Smooth Muscle Phenotype,” Circ. Res. 87:355-362 (2000); Neuville et al., “Retinoids and Arterial Smooth Muscle Cells,” Arterioscler. Thromb. Vasc. Biol. 20:1882-1888 (2000), which are hereby incorporated by reference in their entirety). The underlying mechanisrns of atRA's effects in SMC, however, are currently not well understood although it is presumed that much of the biological activity relates to retinoid receptor-mediated changes in gene expression. Thus, identifying retinoid-response genes in SMC represents a fulfill endeavor towards understanding the biology of retinoids in both SMC and the vessel wall. As disclosed in Example 1 herein, several retinoid-responsive genes in atRA-stimulated SMC have been cloned (Chen et al., “A Novel Retinoid-Response Gene Set in Vascular Smooth Muscle Cells,” Biochem. Biophys. Res. Commun. 281:475-482 (2001), which is hereby incorporated by reference in its entirety). One of the genes cloned, tissue transglutaminase, appears to mediate retinoid-induced SMC apoptosis in vitro (Ou et al., “Retinoic Acid-Induced Tissue Transglutaminase and Apoptosis in Vascular Smooth Muscle Cells,” Circ. Res. 87:881-887 (2000), which is hereby incorporated by reference in its entirety). Other genes cloned included those associated with growth inhibition and SMC differentiation (Chen et al., “A Novel Retinoid-Response Gene Set in Vascular Smooth Muscle Cells,” Biochem. Biophys. Res. Commun. 281:475-482 (2001), which is hereby incorporated by reference in its entirety). In addition, the RISC gene has been identified as having significant sequence homology to several critical domains found in serine carboxypeptidases.

The RISC gene identified herein as SEQ ID No: 36 has significant sequence homology to several critical domains found in serine carboxypeptidases. Several carboxypeptidases have been described in vascular SMC where they function to either activate or inactivate proteins involved with vasomotion or growth (Takai et al., “Different Angiotensin II-Forming Pathways in Human and Rat Vascular Tissues,” Clinica Chimica Acta 305:191-195 (2001); Mentlein et al., “Proteases Involved in the Metabolism of Angiotensin II, Bradykinin, Calcitonin Gene-Related Peptide (CGRP), and Neuropeptide Y by Vascular Smooth Muscle Cells,” Peptides 17:709-720 (1996); Reznik et al., “Immunohistochemical Localization of Carboxypeptidases E and D in the Human Placenta and Umbilical Cord,” J. Histochem. Cytochem. 46:1359-1367 (1998), which are hereby incorporated by reference in their entirety). An aortic carboxypeptidase-like protein was shown to be up-regulated during vascular SMC differentiation in vitro (Layne et al., “Aortic Carboxypeptidase-Like Protein, a Novel Protein With Discoidin and Carboxypeptidase-Like Domains, is Up-Regulated During Vascular Smooth Muscle Cell Differentiation,” J. Biol. Chem. 273:15654-15660 (1998), which is hereby incorporated by reference in its entirety). The majority of SC are found in the plant kingdom where they function in the normal turnover of proteins and the cleavage of amino acids for nutrition (Remington et al., “Carboxypeptidases C and D,” Methods Enzymol. 244:231-248 (1994), which is hereby incorporated by reference in its entirety). A yeast SC, called KEX1, functions as a membrane-associated protease involved in the processing of precursors to secreted mature proteins (Cooper et al., “Characterization of the Yeast KEX1 Gene Product: A Carboxypeptidase Involved in Processing Secreted Precursor Proteins,” Mol. Cell. Biol. 9:2706-2714 (1989), which is hereby incorporated by reference in its entirety).

The induction of RISC mRNA by atRA occurs in a time dependent manner. In normally cultured RASMC, RISC mRNA expression is low. Twelve hours following atRA stimulation there is a detectable increase in RISC mRNA. This increase appears to peak around 4 days following stimulation. At least some of this induction is dependent on de novo protein synthesis as CHX blocked increased RISC expression 48 hr following atRA treatment. No change in RISC expression was observed 24 hours following treatment, suggesting that the RISC mRNA has a long half-life or increase in expression at this time is independent of protein synthesis. Analysis of the tissue expression of RISC revealed that it is highly expressed in the aorta, bladder, and kidney of the rat; high level expression was also observed in human kidney. The restricted expression of RISC to cuboidal epithelial cells of the proximal convoluted tubule is suggestive of a function unique to these highly metabolic cells. These cells appear to be major sites of amino acid reabsorption. Interestingly, endothelin has been shown to be inactivated in the kidney by a protease with structural properties similar to RISC (Deng et al., “A Soluble Protease Identified From Rat Kidney Degrades Endothelin-1 But Not Proendothelin-1,” J. Biochem. 112:168-172 (1992), which is hereby incorporated by reference in its entirety). It will be interesting to determine whether this protease is in fact RISC. Given the physiological differences between SMC and the proximal convoluted tubule epithelium, RISC may very likely display disparate functions in these two cell types.

### Example 3

### RISC Expression in Stably Transfected PAC1 SMC and Effect on SMC Growth

Rat or human RISC is cloned into a suitable mammalian expression plasmid including, but not limited to, pEF1/V5-His, and verified for expression by Western blotting (using an antibody that binds the His tag of the RISC-His fusion protein) of transiently-transfected cells. For transient transfection, subconfluent cells are overlayed with a DNA-calcium-phosphate coprecipitate containing the RISC expression plasmid and then allowed to incubate overnight The following day, cells are washed with phosphate-buffered saline and re-fed fresh growth medium for an additional 24-48 hours whereupon standard cell lysates are made for protein analysis via Western blotting. Once verification of RISC expression is so demonstrated, stable cell lines are generated by repeating the same transfection procedure described above followed by trypsinization and re-plating in the presence of G418 (a neomycin-like analog that normally kills cells without the resistance gene, neomycin, contained within the plasmid vector). Following two weeks of selection in G418, resistant cell “clones” cartying the RISC-His fusion protein are isolated and expanded for further analysis.

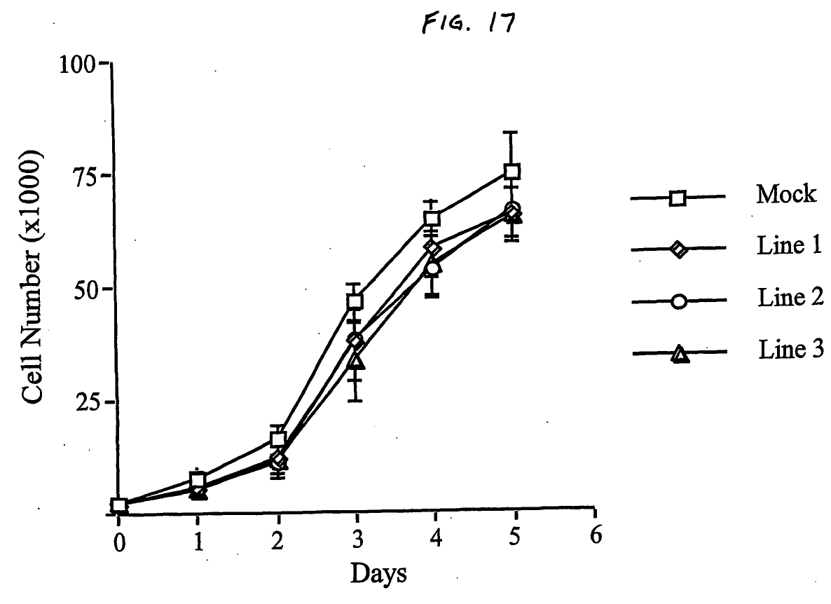

Each of three stably transfected cell lines were grown in the presence of 10% FBS for 5 days and on each day total cell numbers were calculated with a hemocytometer. In all three RISC cell lines, significant inhibition in cell growth was noted as compared to a cell line stably transfected with the empty vector as a control (mock). A graph illustrating the effects on SMC growth is shown at FIG. 17.

### Example 4

### RISC Attenuation of ERK Activity

Stable cell lines prepared according to Example 3 were grown to subconfluence and made quiescent by serum withdrawal (typically 0.5% FBS for 24-48 hours). Cells were then stimulated with serum or a purified growth factor (PDGF-BB) to stimulate the ERK pathway. At selected times following such stimulation, standard cell extracts were prepared to analyze the activation of ERK as measured by its phosphorylated state with a phospho-specific antibody.

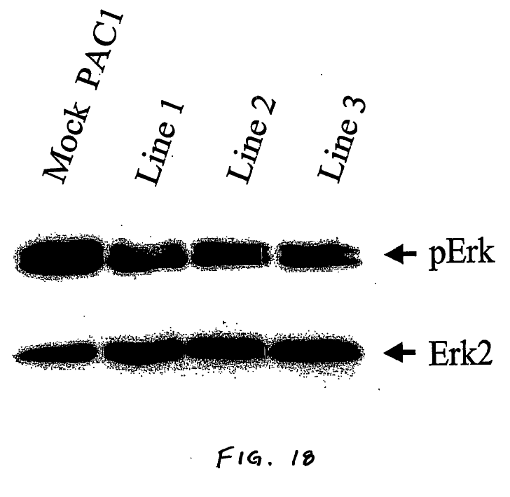

Results showed that RISC expressing cells (as compared to mock cells) activated less phospho-ERK 30 minutes after serum stimulation (FIG. 18). These results are in line with the growth suppression data and suggest that RISC may be cleaving either a growth factor or receptor to attenuate pERK activity.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An isolated mammalian retinoid inducible serine carboxypeptidase protein or polypeptide.

2. The isolated protein or polypeptide according to claim 1, wherein the protein or polypeptide comprises one or more domains selected from the group consisting of:
(a) a serine carboxypeptidase substrate binding domain comprising an amino acid sequence of WXXGGPGXSS (SEQ ID No: 7) where X is any amino acid;
(b) a first catalytic domain comprising an amino acid sequence of XXXESYXG (SEQ ID No: 15) where X is any amino acid;
(c) a second catalytic domain comprising an amino acid sequence of XXXGXXDLI (SEQ ID No: 24) where X is any amino acid; and
(d) a third catalytic domain comprising an amino acid sequence of GXG H (SEQ ID No: 34) where X is any amino acid.

3. The isolated protein or polypeptide according to claim 2, wherein the protein or polypeptide comprises domains (a), (b), (c), and (d).

4. The isolated protein or polypeptide according to claim 2, wherein the serine carboxypeptidase substrate binding domain comprises an amino acid sequence of WLQGGPGGSS (SEQ ID No: 1).

5. The isolated protein or polypeptide according to claim 2, wherein the first catalytic domain comprises an amino acid sequence of IFSESYGG (SEQ ID No: 8).

6. The isolated protein or polypeptide according to claim 2, wherein the second catalytic domain comprises an amino acid sequence of VYNGQLDLI (SEQ ID No: 16).

7. The isolated protein or polypeptide according to claim 2, wherein the third catalytic domain comprises an amino acid sequence of LAFYWILKAGHMVPXDQG (SEQ ID No: 35) where X is A or S.

8. The isolated protein or polypeptide according to claim 2, wherein the protein or polypeptide is isolated from rat or mouse.

9. The isolated protein or polypeptide according to claim 8, wherein the protein or polypeptide has an amino acid sequence of SEQ ID No: 36 or SEQ ID No: 38.

10. The isolated protein or polypeptide according to claim 1, wherein the protein or polypeptide is substantially purified.

11. The isolated protein or polypeptide according to claim 1, wherein the protein or polypeptide is recombinant.

12. An isolated nucleic acid molecule encoding a retinoid inducible serine carbokypeptidase protein or polypeptide according to claim 1.

13. The isolated nucleic acid molecule according to claim 12, wherein the nucleic acid molecule encodes a protein or polypeptide having an amino acid sequence of SEQ ID No: 36 or SEQ ID No: 38.

14. The isolated nucleic acid molecule according to claim 13, wherein the nucleic acid molecule has a nucleotide sequence of SEQ ID No: 37 or SEQ ID No: 39.

15. The isolated nucleic acid molecule according to claim 12, wherein the nucleic acid hybridizes to the complement of a DNA molecule having a nucleotide sequence of SEQ ID No: 36 or SEQ ID No: 38 under stringency conditions comprising a hybridization buffer which includes about 5×SSC and a temperature of about 56° C.

16. The isolated nucleic acid molecule according to claim 12, wherein the nucleic acid is RNA.

17. The isolated nucleic acid molecule according to claim 12, wherein the nucleic acid is DNA.

18. A DNA construct comprising:
a DNA molecule according to claim 17;
a promoter operably linked 5′ of the DNA molecule; and
a 3′ regulatory region operably linked 3′ of the DNA molecule.

19. The DNA construct according to claim 18, wherein the DNA molecule is in a sense orientation relative to the promoter.

20. The DNA construct according to claim 18, wherein the DNA molecule is in an antisense orientation relative to the promoter.

21. An expression vector into which has been inserted a DNA construct according to claim 18.

22. An expression vector into which has been inserted a DNA molecule according to claim 17.

23. A host cell transformed with a DNA molecule according to claim 17.

24. The host cell according to claim 23, wherein the host cell is a bacterial cell, a yeast cell, or a mammalian cell.

25. The host cell according to claim 23, wherein the host cell is in vivo.

26. The host cell according to claim 23, wherein the host cell is in vitro.

27. A method of detecting presence, absence, or changes in progression or regression of a vascular disease or disorder in a subject comprising:
contacting a tissue or fluid sample from a subject with a nucleic acid molecule according to claim 12, or a fragment thereof, as a primer or a probe in a gene amplification detection procedure; and
detecting any reaction which indicates amplification of a target with the primer or probe, where amplification of the target indicates the presence of a vascular disease or disorder and the lack thereof indicates the absence of the vascular disease or disorder.

28. The method according to claim 27, wherein the vascular disease or disorder is a hyperplasia, atherosclerosis, restenosis, glomerulonephritides, hypertension, obstructive bladder disease, tubulosclerosis, asthma, or interstitial tubular disease.

29. The method according to claim 27, wherein the target is an mRNA molecule encoding a retinoid inducible serine carboxypeptidase protein or polypeptide.

30. The method according to claim 27, wherein the nucleic acid molecule is selected from the group consisting of: SEQ ID No: 42, SEQ ID No: 43, SEQ ID No: 44, SEQ ID No: 45, SEQ ID No: 46, SEQ ID No: 47, SEQ ID No: 48, SEQ ID No: 49, SEQ ID No: 50, SEQ ID No: 51, SEQ ID No: 52, SEQ ID No: 53, SEQ ID No: 54, SEQ ID No: 55, SEQ ID No: 56, SEQ ID No: 57, SEQ ID No: 58, SEQ ID No: 59, SEQ ID No: 60, SEQ ID No: 61, SEQ ID No: 62, SEQ ID No: 63, SEQ ID No: 64, and SEQ ID No: 65.

31. A method of detecting presence, absence, or changes in progression or regression of a vascular disease or disorder in a subject, comprising:
contacting a tissue or fluid sample from a subject with a nucleic acid molecule according to claim 12, or a fragment thereof, as a probe under conditions effective to cause hybridization between the probe and a target to form a hybridization complex; and
determining whether any hybridization complex forms during said contacting, where formation of a hybridization complex indicates the presence of a vascular disease or disorder and lack thereof indicates the absence of the vascular disease or disorder.

32. The method according to claim 31, wherein the vascular disease or disorder is a hyperplasia, atherosclerosis, restenosis, glomerulonephritides, hypertension, obstructive bladder disease, tubulosclerosis, asthma, or interstitial tubular disease.

33. The method according to claim 31, wherein the target is an mRNA molecule encoding a retinoid inducible serine carboxypeptidase protein or polypeptide.

34. The method according to claim 31 further comprising:
washing the contacted tissue or fluid sample to remove probe not bound to the target prior to said determining.

35. The method according to claim 31, wherein the nucleic acid molecule is selected from a group consisting of: SEQ ID No: 42, SEQ ID No: 43, SEQ ID No: 44, SEQ ID No: 45, SEQ ID No: 46, SEQ ID No: 47, SEQ ID No: 48, SEQ ID No: 49, SEQ ID No: 50, SEQ ID No: 51, SEQ ID No: 52, SEQ ID No: 53, SEQ ID No: 54, SEQ ID No: 55, SEQ ID No: 56, SEQ ID No: 57, SEQ ID No: 58, SEQ ID No: 59, SEQ ID No: 60, SEQ ID No: 61, SEQ ID No: 62, SEQ ID No: 63, SEQ ID No: 64, and SEQ ID No: 65.

36. An isolated antibody or binding portion thereof which binds to a protein or polypeptide according to claim 1.

37. The isolated antibody or binding portion thereof according to claim 36, wherein the antibody or binding portion thereof is monoclonal or polyclonal.

38. The isolated antibody or binding portion thereof according to claim 36, wherein the antibody or binding portion thereof binds to a domain of the protein or polypeptide selected from the group consisting of:
(a) a serine carboxypeptidase substrate binding domain comprising an amino acid sequence of WXXGGPGXSS (SEQ ID No: 7) where X is any amino acid;
(b) a first catalytic domain comprising an amino acid sequence of XXXESYXG (SEQ ID No: 15) where X is any amino acid;
(c) a second catalytic domain comprising an amino acid sequence of XXXGXXDLI (SEQ ID No: 24) where X is any amino acid; and
(d) a third catalytic domain comprising an amino acid sequence of XXXXXXXXXGHMXXXXXX (SEQ ID No: 34) where X is any amino acid.

39. The isolated antibody or binding portion thereof according to claim 36, wherein the antibody or binding portion thereof binds to a protein or polypeptide having an amino acid sequence of SEQ ID No: 36 or SEQ ID No: 38 or SEQ ID No: 40.

40. The isolated antibody or binding portion thereof according to claim 36, wherein the antibody or binding portion thereof neutralizes activity of the mammalian retinoid inducible serine carboxypeptidase.

41. A method of detecting presence, absence, or changes in progression or regression of a vascular disease or disorder in a subject comprising:
contacting a tissue or fluid sample from a subject with an antibody or binding portion according to claim 36 under conditions effective to permit formation of an antigen-antibody/binding portion complex; and
determining whether the antigen-antibody/binding portion complex has formed using an assay system, where the presence of antigen-antibody/binding portion complex indicates the presence of a vascular disease or disorder and the lack thereof indicates the absence of the vascular disease or disorder.

42. The method according to claim 41, wherein the assay system is selected from the group consisting of an enzyme-linked immunosorbent assay, a radioimmunoassay, a gel diffusion precipitin reaction assay, an immunodiffusion assay, an agglutination assay, a fluorescent immunoassay, a protein A immunoassay, and an immunoelectrophoresis assay.

43. The method according to claim 41, wherein the vascular disease or disorder is a hyperplasia, atherosclerosis, restenosis, glomerulonephritides, hypertension, obstructive bladder disease, tubulorsclerosis, asthma, or interstitial tubular disease.

44. The method according to claim 41 further comprising:
washing the contacted tissue or fluid sample to remove antibody or binding portions thereof which have not formed an antigen-antibody/binding portion complex prior to said determining.

45. A method of inhibiting smooth muscle cell growth comprising:
increasing the intracellular concentration of a retinoid-inducible protein or polypeptide in a smooth muscle cell under conditions effective to inhibit growth of the smooth muscle cell.

46. The method according to claim 45, wherein the retinoid-inducible protein or polypeptide is retinoid inducible serine carboxypeptidase, spermidine/spermine N-acetyltransferase, src suppressed C kinase substrate, epithelin, α8-integrin, vascular cell adhesion molecule-1, tissue transglutaminase, lactate dehydrogenase-B, lectin-like oxidized LDL receptor, retinol dehydrogenase, cathepsin-L, ceruloplasmin, importin α, endolyn, stoned B/TFIIAα/β-like factor, or a combination thereof.

47. The method according to claim 45, wherein said increasing comprises:
introducing the retinoid-inducible protein or polypeptide into the smooth muscle cell.

48. The method according claim 47, wherein said introducing comprises:
contacting the smooth muscle cell with a delivery vehicle comprising the retinoid-inducible protein or polypeptide.

49. The method according to claim 48, wherein the delivery vehicle is a fusion protein comprising the retinoid-inducible protein or polypeptide.

50. The method according to claim 48, wherein the delivery vehicle is a liposome.

51. The method according to claim 48, wherein the delivery vehicle is an enzymatically stable conjugate comprising a polymer and the retinoid-inducible protein or polypeptide conjugated to the polymer.

52. The method according to claim 48, wherein the delivery vehicle is a catheter comprising the retinoid-inducible protein or polypeptide.

53. The method according to claim 45, wherein said increasing comprises:
transforming the smooth muscle cell with a nucleic acid encoding the retinoid-inducible protein or polypeptide under conditions effective for expression of the retinoid-inducible protein or polypeptide in the transformed smooth muscle cell.

54. The method according to claim 53, wherein said transforming comprises:
transforming the smooth muscle cell with an infective transformation vector comprising the nucleic acid encoding the retinoid-inducible protein or polypeptide.

55. The method according to claim 54, wherein the infective transformation vector is an adenovirus vector, a retrovirus vector, or a lentivirus vector.

56. The method according to claim 45, wherein the smooth muscle cell is in vivo.

57. A method of treating vascular hyperplasia comprising:
increasing the intracellular concentration of a retinoid-inducible protein or polypeptide in vascular smooth muscle cells at a site of vascular hyperplasia under conditions effective to treat the vascular hyperplasia.

58. The method according to claim 57, wherein the retinoid-inducible protein or polypeptide is retinoid inducible serine carboxypeptidase, spermidine/spermine N-acetyltransferase, src suppressed C kinase substrate, epithelin, α₈-integrin, vascular cell adhesion molecule-1, tissue transglutaminase, lactate dehydrogenase-B, lectin-like oxidized LDL receptor, retinol dehydrogenase, cathepsin-L, ceruloplasmin, importin a, endolyn, stoned B/TFIIAα/β-like factor, or a combination thereof.

59. The method according to claim 57, wherein said increasing comprises:
introducing the retinoid-inducible protein or polypeptide into one or more smooth muscle cells at the site of vascular hyperplasia.

60. The method according claim 59, wherein said introducing comprises:
contacting the one or more smooth muscle cells with a delivery vehicle comprising the retinoid-inducible protein or polypeptide.

61. The method according to claim 60, wherein the delivery vehicle is a fision protein comprising the retinoid-inducible protein or polypeptide.

62. The method according to claim 60, wherein the delivery vehicle is a liposome.

63. The method according to claim 60, wherein the delivery vehicle is an enzymatically stable conjugate comprising a polymer and the retinoid-inducible protein or polypeptide conjugated to the polymer.

64. The method according to claim 60, wherein the delivery vehicle is a catheter comprising the retinoid-inducible protein or polypeptide.

65. The method according to claim 57, wherein said increasing comprises:
transforming the one or more smooth muscle cells with a nucleic acid encoding the retinoid-inducible protein or polypeptide under conditions effective for expression of the retinoid-inducible protein or polypeptide in the transformed one or more smooth muscle cells.

66. The method according to claim 65, wherein said transforming comprises:
introducing an infective transformation vector comprising the nucleic acid encoding the retinoid-inducible protein or polypeptide into the one or more smooth muscle cells.

67. The method according to claim 66, wherein the infective transformation vector is an adenovirus vector, a retrovirus vector, or a lentivirus vector.

68. A method of inhibiting the activity of extracellular regulated kinase comprising:
contacting an extracellular regulated kinase with a retinoid-inducible protein or polypeptide under conditions effective to inhibit the activity of the extracellular regulated kinase.

69. The method according to claim 68, wherein said contacting inhibits the phosphorylation of the extracellular regulated kinase.

70. A transgenic non-human animal whose somatic and germ cells lack a gene encoding a retinoid inducible protein or polypeptide, or possess a disruption in that gene, whereby the animal exhibits increased smooth muscle cell growth and neointimal formation following vascular trauma as compared to non-transgenic animals.

71. The transgenic non-human animal according to claim 70, wherein the animal is selected from the group consisting of mouse and rat.

72. The transgenic non-human animal according to claim 70, wherein the retinoid-inducible protein or polypeptide is a retinoid inducible serine carboxypeptidase protein or polypeptide.

73. The transgenic non-human animal according to claim 70, wherein the transgenic non-human animal further comprises a lacZ reporter gene.


SEQUENCE LISTING (70 sequences)

SEQ ID NO: 1 - protein, 10 aa, Artificial Sequence
  Description of Artificial Sequence  serine carboxypeptidase substrate binding domain I of human, rat, and mouse RISC
WLQGGPGGSS

SEQ ID NO: 2 - protein, 10 aa, Artificial Sequence
  Description of Artificial Sequence  serine carboxypeptidase substrate binding domain I of C. elegans F22E12.1
WLQGGPGSSS

SEQ ID NO: 3 - protein, 10 aa, Artificial Sequence
  Description of Artificial Sequence  serine carboxypeptidase substrate binding domain I of D. melanogaster CG3344 and A. aegypti VCP
WLQGGPGASS

SEQ ID NO: 4 - protein, 10 aa, Artificial Sequence
  Description of Artificial Sequence  serine carboxypeptidase substrate binding domain I of human protective protein (cathepsin A) and S. cerevisiae Cpd Y
WLNGGPGCSS

SEQ ID NO: 5 - protein, 10 aa, Artificial Sequence
  Description of Artificial Sequence  serine carboxypeptidase substrate binding domain I of C. elegans F41C3.5
WFNGGPGCSS

SEQ ID NO: 6 - protein, 10 aa, Artificial Sequence
  Description of Artificial Sequence  serine carboxypeptidase substrate binding domain I of H. vulgare Cpd C
WLTGGPGCSS

SEQ ID NO: 7 - protein, 10 aa, Artificial Sequence
  Description of Artificial Sequence  consensus sequence for sequences 1-6
WXXGGPGXSS

SEQ ID NO: 8 - protein, 8 aa, Artificial Sequence
  Description of Artificial Sequence  catalytic domain II of human, rat, and mouse RISC
IFSESYGG

SEQ ID NO: 9 - protein, 8 aa, Artificial Sequence
  Description of Artificial Sequence  catalytic domain II of C. elegans F22DE12.1 and D. melanogaster CG3344
IFCESYGG

SEQ ID NO: 10 - protein, 8 aa, Artificial Sequence
  Description of Artificial Sequence  catalytic domain II of A. aegypti VCP
ISGESYGG

SEQ ID NO: 11 - protein, 8 aa, Artificial Sequence
  Description of Artificial Sequence  catalytic domain II of human protective protein (cathepsin A)
LTGESYAG

SEQ ID NO: 12 - protein, 8 aa, Artificial Sequence
  Description of Artificial Sequence  catalytic domain II of C. elegans F41C3.5
IMGESYGG

SEQ ID NO: 13 - protein, 8 aa, Artificial Sequence
  Description of Artificial Sequence  catalytic domain II of H. vulgare Cpd C
ITGESYAG

SEQ ID NO: 14 - protein, 8 aa, Artificial Sequence
  Description of Artificial Sequence  catalytic domain II of S. cerevisiae Cpd Y
IAGESYAG

SEQ ID NO: 15 - protein, 8 aa, Artificial Sequence
  Description of Artificial Sequence  consensus sequence for sequences 8-14
XXXESYXG

SEQ ID NO: 16 - protein, 9 aa, Artificial Sequence
  Description of Artificial Sequence  catalytic domain III of human, rat, and mouse RISC
VYNGQLDLI

SEQ ID NO: 17 - protein, 9 aa, Artificial Sequence
  Description of Artificial Sequence  catalytic domain III of C. elegans F22E12.1
VYNGNEDLI

SEQ ID NO: 18 - protein, 9 aa, Artificial Sequence
  Description of Artificial Sequence  catalytic domain III of D. melanogaster CG3344
VFSGGLDLI

SEQ ID NO: 19 - protein, 9 aa, Artificial Sequence
  Description of Artificial Sequence  catalytic domain III of A. aegypti VCP
FYNGQLDII

SEQ ID NO: 20 - protein, 9 aa, Artificial Sequence
  Description of Artificial Sequence  catalytic domain III human protective protein (cathepsin A)
LYNGDVDMA

SEQ ID NO: 21 - protein, 9 aa, Artificial Sequence
  Description of Artificial Sequence  catalytic domain III of C. elegans F41C3.5
LYYGDTDMA

SEQ ID NO: 22 - protein, 9 aa, Artificial Sequence
  Description of Artificial Sequence  catalytic domain III of H. vulgare Cpd C
IYAGEYDLI

SEQ ID NO: 23 - protein, 9 aa, Artificial Sequence
  Description of Artificial Sequence  catalytic domain III of S. cerevisiae Cpd Y
VYAGDKDFI

SEQ ID NO: 24 - protein, 9 aa, Artificial Sequence
  Description of Artificial Sequence  consensus sequence for sequences 16-23
XXXGXXDLI

SEQ ID NO: 25 - protein, 18 aa, Artificial Sequence
  Description of Artificial Sequence  catalytic domain III of rat RISC
LAFYWILKAGHMVPADQG

SEQ ID NO: 26 - protein, 18 aa, Artificial Sequence
  Description of Artificial Sequence  catalytic domain III of human and mouse RISC
LAFYWILKAGHMVPSDQG

SEQ ID NO: 27 - protein, 18 aa, Artificial Sequence
  Description of Artificial Sequence  catalytic domain III of C. elegans F22E12.1
LQFWWILRAGHMVAYDTP

SEQ ID NO: 28 - protein, 18 aa, Artificial Sequence
  Description of Artificial Sequence  catalytic domain III of D. melanogaster CG3344
FSMFWVNRAGHMVPADNP

SEQ ID NO: 29 - protein, 18 aa, Artificial Sequence
  Description of Artificial Sequence  catalytic domain III of A. aegypti VCP
LQEVLIRNAGHMVPRDQP

SEQ ID NO: 30 - protein, 18 aa, Artificial Sequence
  Description of Artificial Sequence  catalytic domain III of human protective protein (cathepsin A)
IAFLTIKGAGHMVPTDKP

SEQ ID NO: 31 - protein, 18 aa, Artificial Sequence
  Description of Artificial Sequence  catalytic domain III of C. elegans F41C3.5
LSFITIRGAGHMAPQWRA

SEQ ID NO: 32 - protein, 18 aa, Artificial Sequence
  Description of Artificial Sequence  catalytic domain III of H. vulgare Cpd C
LSFLKVHNAGHMVPMDQP

SEQ ID NO: 33 - protein, 18 aa, Artificial Sequence
  Description of Artificial Sequence  catalytic domain III of S. cerevisiae Cpd Y
FTYLRVFNGGHMVPFDVP

SEQ ID NO: 34 - protein, 18 aa, Artificial Sequence
  Description of Artificial Sequence  consensus sequence for sequences 15-33
XXXXXXXXXGHMXXXXXX

SEQ ID NO: 35 - protein, 18 aa, Artificial Sequence
  Description of Artificial Sequence  consensus sequence for sequences 25-26
LAFYWILKAGHMVPXDQG

SEQ ID NO: 36 - protein, 452 aa, Rattus norvegicus
MELSRRICLVRLWLLLLSFLLGFSAGSALNWREQEGKEVWDYVTVREDARMFWWLYYATNPCKNFSELPLVMWLQGGPGGSSTGFGNFEEIGPLDTRLKPRNTTWLQWASLLFVDNPVGTGFSYVNTTDAYAKDLDTVASDMMVLLKSFFDCHKEFQTVPFYIFSESYGGKMAAGISLELHKAIQQGTIKCNFSGVALGDSWISPVDSVLSWGPYLYSVSLLDNKGLAEVSDIAEQVLNAVNKGFYKEATQLWGKAEMIIEKNTDGVNFYNILTKSTPDTSMESSLEFFRSPLVRLCQRHVRHLQGDALSQLMNGPIKKKLKIIPDDVSWGAQSSSVFISMEEDFMKPVIDIVDTLLELGVNVTVYNGQLDLIVDTIGQESWVQKLKWPQLSRFNQLKWKALYTNPKSSETSAFVKSYENLAFYWILKAGHMVPADQGDMALKMMRLVTQQE

SEQ ID NO: 37 - DNA, 2046 bp, Rattus norvegicus
ctgaggcggggttttcatcatggagctgtcgcggcggatttgtctcgtccgactgtggctgttgctactgtcgttcttgctgggcttcagcgcgggatctgccctcaactggcgggaacaagaaggcaaggaagtatgggattacgtgactgttcgagaggatgcacgcatgttctggtggctctactatgccaccaacccttgcaagaacttctcagagctgcctctggtcatgtggcttcagggtggtccaggtggttctagcactggatttggaaactttgaggaaatcggccctcttgacacccgactcaagccacggaacactacctggctgcagtgggccagtctcctgtttgtggacaatcctgtgggcacgggcttcagttacgtgaacacgacagatgcctacgcaaaggacctggacacggtggcttccgacatgatggtcctcctgaaatccttctttgattgtcataaagaattccagacggttccgttctacattttctcagaatcctacggaggaaagatggctgctggcatcagtttagaacttcacaaggctattcagcaagggaccatcaagtgcaacttctctggggttgctttgggtgactcctggatctcccctgtggattcagtgctgtcctggggaccttacctgtacagcgtgtctctccttgataataaaggcttggctgaggtgtccgacattgcggagcaagtcctcaatgctgtaaacaagggcttctacaaggaagccactcagctgtgggggaaagcagaaatgatcattgaaaagaacaccgacggggtaaacttctataacatcttaactaaaagcacccccgacacctctatggagtcgagcctcgagttcttccggagccccttagttcgtctctgtcagcgccacgtgagacacctacaaggagacgccttaagtcagctcatgaacggtcccatcaaaaagaagctcaaaattatccctgacgatgtctcctggggagcccagtcgtcctccgtcttcataagcatggaagaggacttcatgaagcctgtcatcgacatcgtggatacgttgctggaactcggggtcaatgtgactgtgtacaatgggcagctggatctcattgtggacaccataggtcaggagtcctgggttcagaagctgaagtggccacagctgtccagattcaatcagctaaaatggaaggccctgtacaccaatcctaagtcttcagaaacatctgcgtttgtcaagtcctatgagaacttagcgttctactggatcctaaaggcgggtcacatggttcctgctgaccaaggggacatggctctgaagatgatgaggctggttactcagcaggagtagctgagctgagctggccctggaggccctggaggccctggaggccctggagtagggcccaggatgcaggtgctaatgtctatccccggcgctcttcttcccgactctaccatgggatgtaactccaggagcccctgccatctcccgtaccaaaagactgtggcttccgtgtctactcagaaatcagttctacttcgtaaacagtgtttaaaaccagactcatttaatcagagtgaaggattgcagtccattggcttcttagcacagaagcagctgataacacaagtaaaccccagcccttgagaggtagaagcaagaggatcagaggttcaagcgcatcctcggctccatcataagttcaaaagccgcctgcaccaaatgggagtccttgtctcaaaaaaaaaaaaaaaaaaaaaaaagcaaagaaagcaaaggactcgatgacatgatttatagacaaaagcagtgggagaaaatactaaagccccactgagctgccagccaggtgtctgtgactacaggtcttttatctgctacatatatatttttacaaaaatgaaatccatattgttcgctattttgctgtctgctttgctcccgtatcaacatgacttgcacgtcttttcccatcaataaatgtgccatgatatttttaaaaaaaaaaaaaaaaa

SEQ ID NO: 38 - protein, 452 aa, Mus musculus
MELSRRICLVRLWLLLLSFLLGFSAGSAIDWREPEGKEVWDYVTVRKDAHMFWWLYYATNPCKNFSELPLVMWLQGGPGGSSTGFGNFEEIGPLDTQLKPRNTTWLQWASLLFVDNPVGTGFSYVNTTDAYAKDLDTVASDMMVLLKSFFDCHKEFQTVPFYIFSESYGGKMAAGISVELYKAVQQGTIKCNFSGVALGDSWISPVDSVLSWGPYLYSMSLLDNQGLAMVSDIAEQVLDAVNKGFYKEATQLWGKAEMIIEKNTDGVNFYNILTKSSPEKAMESSLEFLRSPLVRLCQRHVRHLQGDALSQLMNGPIKKKLKIIPEDISWGAQASYVFLSMEGDFMKPAIDVVDKLLAAGVNVTVYNGQLDLIVDTIGQESWVQKLKWPQLSKFNQLKWKALYTDPKSSETAAFVKSYENLAFYWILKAGHMVPSDQGEMALKMMKLVTKQE

SEQ ID NO: 39 - DNA, 2045 bp, Mus musculus
ggttgctgatgttcggcggggttttcatcatggagctctcgcggcggatctgtctcgtgcgactgtggctgctgctcctatcgttcttactgggcttcagcgcgggatctgccatcgactggcgggaacccgaaggcaaggaagtatgggattatgtgactgtccgaaaggatgcccacatgttctggtggctctattatgccaccaacccttgcaagaacttttcagagctgcccctggtcatgtggcttcagggtggtccgggtggttctagcactggatttggaaactttgaggaaatcggccctcttgacacccaactcaagcctcgaaataccacctggctgcagtgggccagtctcctgtttgtggataatcccgtgggcacgggcttcagctacgtcaacacaacagatgcctacgcaaaggacctggacacggtggcttccgacatgatggttctcctgaaatccttctttgattgccataaagaattccagacggttccattctacattttctcagaatcctacggaggaaagatggctgctggcatcagtgtagaactttacaaggctgttcagcaagggaccattaagtgcaacttttctggggttgctttgggtgactcctggatctcccccgtggattcagtgctgtcctggggaccttacctgtatagtatgtctctccttgataatcaaggcttggcgatggtgtccgacattgcagagcaagtcctcgatgctgtaaacaagggcttctacaaggaggccactcagctgtgggggaaagcagaaatgatcattgaaaagaacaccgacggggtaaacttctataacatcttaactaaaagcagcccggagaaagctatggaatcgagcctcgagttcctccggagccccttagttcgtctctgtcagcgccatgtgagacacctgcaaggagacgccttaagtcaactcatgaacggccccatcaaaaagaagctcaaaattatccctgaggatatctcctggggagcccaggcatcttatgtcttcctaagcatggaaggggacttcatgaagcctgccatcgacgttgtggataagttgctggcagctggggtcaatgtgaccgtgtacaacggacagctggatctcattgtggacaccataggtcaggagtcctgggttcagaagctcaagtggccacagctgtccaaattcaatcagctaaaatggaaggccctgtacaccgatcctaagtcttcagaaacagctgcgttcgtcaagtcctatgagaacctagccttctactggatcctaaaggccggtcacatggttccttctgaccaaggggagatggccctgaagatgatgaagctggtgaccaagcaggagtagctgagctggctggccctggaggcgctaagagcagagcccagaatgcaggtgctaatgtctatccctggtgctcttctcccctgctctgccatgggatatgactctgggagcacctgctctctcccgtaccgaaagactgtggccttctgtgtctacttagaaatcagttctgcttcccaaagagtatttaaaaccagactcatttaatcaaagtgaagggttgcaatcgattggtctcttactacaaaagcagttgatagcacatgtaaatccaagcacttgagaggtagaagaagcaagaggatggatgagaggttcaaacgcatccgcagctacatcgaaagttcaaaagcagcctatgccaaacagggagagtccctgtcccccacccccacccccaaaaaagagcaaaagcaaaccacatgatttatagacaaaagcagtgggagagacaaagaaaatacttaaacacccactgagctgccaactaggtgtctctgactacgggtcttttatttgctacatatatatttttacaaaaatgaaatccatattgtacgctattttgctgtctgcttcgttcccatgtcgacatgacccgcacttcttttcccatcaataaatgtgttgtgatatttttaaaaaaaaaaaaaaaaa

SEQ ID NO: 40 - protein, 452 aa, Homo sapiens
MELALRRSPVPRWLLLLPLLLGLNAGAVIDWPTEEGKEVWDYVTVRKDAYMFWWLYYATNSCKNFSELPLVMWLQGGPGGSSTGFGNFEEIGPLDSDLKPRKTTWLQAASLLFVDNPVGTGFSYVNGSGAYAKDLAMVASDMMVLLKTFFSCHKEFQTVPFYIFSESYGGKMAAGIGLELYKAIQRGTIKCNFAGVALGDSWISPVDSVLSWGPYLYSMSLLEDKGLAEVSKVAEQVLNAVNKGLYREATELWGKAEMIIEQNTDGVNFYNILTKSTPTSTMESSLEFTQSHLVCLCQRHVRHLQRDALSQLMNGPIRKKLKIIPEDQSWGGQATNVFVNMEEDFMKPVISIVDELLEAGINVTVYNGQLDLIVDTMGQEAWVRKLKWPELPKFSQLKWKALYSDPKSLETSAFVKSYKNLAFYWILKAGHMVPSDQGDMALKMMRLVTQQE

SEQ ID NO: 41 - DNA, 1921 bp, Homo sapiens
cctgttgctgatgctgccgtgcggtacttgtcatggagctggcactgcggcgctctcccgtcccgcggtggttgctgctgctgccgctgctgctgggcctgaacgcaggagctgtcattgactggcccacagaggagggcaaggaagtatgggattatgtgacggtccgcaaggatgcctacatgttctggtggctctattatgccaccaactcctgcaagaacttctcagaactgcccctggtcatgtggcttcagggcggtccaggcggttctagcactggatttggaaactttgaggaaattgggccccttgacagtgatctcaaaccacggaaaaccacctggctccaggctgccagtctcctatttgtggataatcccgtgggcactgggttcagttatgtgaatggtagtggtgcctatgccaaggacctggctatggtggcttcagacatgatggttctcctgaagaccttcttcagttgccacaaagaattccagacagttccattctacattttctcagagtcctatggaggaaaaatggcagctggcattggtctagagctttataaggccattcagcgagggaccatcaagtgcaactttgcgggggttgccttgggtgattcctggatctcccctgttgattcggtgctctcctggggaccttacctgtacagcatgtctcttctcgaagacaaaggtctggcagaggtgtctaaggttgcagagcaagtactgaatgccgtaaataaggggctctacagagaggccacagagctgtgggggaaagcagaaatgatcattgaacagaacacagatggggtgaacttctataacatcttaactaaaagcactcccacgtctacaatggagtcgagtctagaattcacacagagccacctagtttgtctttgtcagcgccacgtgagacacctacaacgagatgccttaagccagctcatgaatggccccatcagaaagaagctcaaaattattcctgaggatcaatcctggggaggccaggctaccaacgtctttgtgaacatggaggaggacttcatgaagccagtcattagcattgtggacgagttgctggaggcagggatcaacgtgacggtgtataatggacagctggatctcatcgtagataccatgggtcaggaggcctgggtgcggaaactgaagtggccagaactgcctaaattcagtcagctgaagtggaaggccctgtacagtgaccctaaatctttggaaacatctgcttttgtcaagtcctacaagaaccttgctttctactggattctgaaagctggtcatatggttccttctgaccaaggggacatggctctgaagatgatgagactggtgactcagcaagaataggatggatggggctggagatgagctggtttggccttggggcacagagctgagctgaggccgctgaagctgtaggaagcgccattcttccctgtatctaactggggctgtgatcaagaaggttctgaccagcttctgcagaggataaaatcattgtctctggaggcaatttggaaattatttctgcttcttaaaaaaacctaagattttttaaaaaattgatttgttttgatcaaaataaaggatgataatagatattattttttcttatgacagaagcaaatgatgtgatttatagaaaaactgggaaatacaggtacccaaagagtaaatcaacatctgtatacccccttcccaggggtaagcactgttaccaatttagcatatgtccttgcagaatttttttttctatatatacatatatattttttaccaaaatgaatcattactctatgttgttttactatttgtttgacatatcagtatatctgaaacaccttttcatgtcaataaatgttcttctctaacattaa

SEQ ID NO: 42 - DNA, 18 bp, Artificial Sequence
  Description of Artificial Sequence  primer
cctgttgctgatgctgcc

SEQ ID NO: 43 - DNA, 18 bp, Artificial Sequence
  Description of Artificial Sequence  primer
ctgcgttcaggcccagca

SEQ ID NO: 44 - DNA, 18 bp, Artificial Sequence
  Description of Artificial Sequence  primer
gagctgtcattgactggc

SEQ ID NO: 45 - DNA, 18 bp, Artificial Sequence
  Description of Artificial Sequence  primer
ctgaagccacatgaccag

SEQ ID NO: 46 - DNA, 18 bp, Artificial Sequence
  Description of Artificial Sequence  primer
ggcggtccaggcggttct

SEQ ID NO: 47 - DNA, 18 bp, Artificial Sequence
  Description of Artificial Sequence  primer
ccaggtggttttccgtgg

SEQ ID NO: 48 - DNA, 18 bp, Artificial Sequence
  Description of Artificial Sequence  primer
ctccaggctgccagtctc

SEQ ID NO: 49 - DNA, 18 bp, Artificial Sequence
  Description of Artificial Sequence  primer
ctggaattctttgtggca

SEQ ID NO: 50 - DNA, 18 bp, Artificial Sequence
  Description of Artificial Sequence  primer
acagttccattctacatt

SEQ ID NO: 51 - DNA, 18 bp, Artificial Sequence
  Description of Artificial Sequence  primer
cttataaagctctagacc

SEQ ID NO: 52 - DNA, 18 bp, Artificial Sequence
  Description of Artificial Sequence  primer
gccattcagcgagggacc

SEQ ID NO: 53 - DNA, 18 bp, Artificial Sequence
  Description of Artificial Sequence  primer
caacaggggagatccagg

SEQ ID NO: 54 - DNA, 18 bp, Artificial Sequence
  Description of Artificial Sequence  primer
cgaagacaaaggtctggc

SEQ ID NO: 55 - DNA, 18 bp, Artificial Sequence
  Description of Artificial Sequence  primer
ttctgctttcccccacag

SEQ ID NO: 56 - DNA, 20 bp, Artificial Sequence
  Description of Artificial Sequence  primer
aacacagatggggtgaactt

SEQ ID NO: 57 - DNA, 18 bp, Artificial Sequence
  Description of Artificial Sequence  primer
ctaggtggctctgtgtga

SEQ ID NO: 58 - DNA, 20 bp, Artificial Sequence
  Description of Artificial Sequence  primer
tttgtctttgtcagcgccac

SEQ ID NO: 59 - DNA, 18 bp, Artificial Sequence
  Description of Artificial Sequence  primer
ggcctccccaggattgat

SEQ ID NO: 60 - DNA, 18 bp, Artificial Sequence
  Description of Artificial Sequence  primer
ccaggctaccaacgtctt

SEQ ID NO: 61 - DNA, 18 bp, Artificial Sequence
  Description of Artificial Sequence  primer
gacccatggtatctacga

SEQ ID NO: 62 - DNA, 19 bp, Artificial Sequence
  Description of Artificial Sequence  primer
cctgggtgcggaaactgaa

SEQ ID NO: 63 - DNA, 19 bp, Artificial Sequence
  Description of Artificial Sequence  primer
gaccagctttcagaatcca

SEQ ID NO: 64 - DNA, 18 bp, Artificial Sequence
  Description of Artificial Sequence  primer
cttctgaccaaggggaca

SEQ ID NO: 65 - DNA, 25 bp, Artificial Sequence
  Description of Artificial Sequence  primer
gttagagaagaacatttattgacat

SEQ ID NO: 66 - DNA, 30 bp, Artificial Sequence
  Description of Artificial Sequence  primer
gatacgtcgacctgaggcggggttttcatc

SEQ ID NO: 67 - DNA, 31 bp, Artificial Sequence
  Description of Artificial Sequence  primer
gatacgatatctgtgatggagccgaggatgc

SEQ ID NO: 68 - DNA, 29 bp, Artificial Sequence
  Description of Artificial Sequence  primer
gatactctagactcctgctgagtaaccag

SEQ ID NO: 69 - DNA, 22 bp, Artificial Sequence
  Description of Artificial Sequence  primer
ctcttcttcccgactctaccat

SEQ ID NO: 70 - DNA, 21 bp, Artificial Sequence
  Description of Artificial Sequence  primer
gaacttgtgatggagccgagg